(19) United States
(12) Patent Application Publication
(10) Pub. No.: US 2023/0009972 A1
(43) Pub. Date: Jan. 12, 2023

(54) METHOD FOR ENRICHING VESICULAR RNA

(71) Applicant: QIAGEN GmbH, Hilden (DE)

(72) Inventors: Martin SCHLUMPBERGER, Hilden (DE); Peter GRÜNEFELD, Hilden (DE); Mark BALTES, Hilden (DE)

(21) Appl. No.: 17/785,251

(22) PCT Filed: Dec. 16, 2020
(86) PCT No.: PCT/EP2020/086585

§ 371 Date: Jun. 14, 2022

(30) Foreign Application Priority Data
Dec. 16, 2019 (EP) 19216752.6

(51) Int. Cl. C12N 15/10

(52) U.S. Cl. CPC C12N 15/101

(57) ABSTRACT

The present invention pertains to methods and kits for enriching extracellular nucleic acids such as vesicular RNA from a sample comprising extracellular vesicles. Accordingly to the methods an acidic binding mixture is prepared comprising the sample and anion exchange particles and binding extracellular vesicles to the anion exchange particles. After separating the anion exchange particles comprising the bound extracellular vesicles from the remaining mixture, bound extracellular vesicles are lysing the in the presence of at least one detergent and released RNA is bound to the anion exchange particles. The anion exchange particles with the bound RNA from the lysate are then eluted.

### FIELD OF THE DISCLOSURE

The present invention pertains to methods and kits for enriching extracellular RNA from a sample comprising extracellular vesicles. In particular, a method is disclosed for the direct enrichment of extracellular nucleic acids such as extracellular RNA from samples comprising extracellular vesicles.

### BACKGROUND

Analytes from extracellular vesicles (EVs), in particular extracellular RNA (also referred to as “cell-free” or “cfRNA” herein) are relevant for diagnostics and research purposes. Anion exchange matrices, such as membranes, can be used to capture EVs from biological samples, in particular from cell-free biofluids, such as plasma, serum, or urine, (see EP 2 941 629 B1 and WO 2017/197399, as well as exoRNeasy kits from QIAGEN). A downside of these binding matrices is that elution is typically performed using high salt concentrations, which renders the eluates incompatible with most downstream applications without further time-consuming and complex cleanup workflows. Alternatively, anion exchange matrices carrying primary, secondary and/or tertiary amino groups as the functional moiety can also be eluted at high pH, usually at pH values higher than 10, which is however detrimental for RNA integrity. Therefore, existing products like exoRNeasy use a combined lysis and elution step using QIAzol, which contains a high salt concentration, such as guanidium thiocyanate or sodium thiocyanate, and phenol. An elution with QIAzol is then followed by organic extraction, in particular using chloroform, and RNA cleanup on silica matrices (e.g. RNeasy columns or MAS G beads).

There is an increasing interest and need for further methods for enriching and thus isolating extracellular RNA from a sample comprising extracellular vesicles. In particular, there is a need for improved methods for isolating extracellular RNA comprising vesicular RNA.

It is an object of the present disclosure to provide a method that avoids drawbacks of the prior art. In particular, it is an object to provide methods for isolating RNA from extracellular vesicles that are less time consuming than prior art workflows. It is furthermore an object to provide a method that avoids laborious and complex clean-up protocols after isolating extracellular RNA such as vesicular RNA.

### SUMMARY

The present disclosure is based on the finding that extracellular vesicles can be bound to anion exchange particles and subsequently be lysed efficiently under conditions that facilitate instant rebinding of the extracellular RNA released that was released from the extracellular vesicles. As a result, the extracellular vesicles do not need to be eluted and extracted using high salt and/or phenol-based elution solution, followed by RNA enrichment protocols. The released vesicular RNA can be directly enriched by binding to anion exchange particles that were used initially for binding the extracellular vesicles. Thereby, a simplified workflow for binding extracellular vesicles (EVs) to anion exchange particles is provided, that is followed by a non-chaotropic lysis of the bound EVs. Immediate rebinding of the released nucleic acid to the same anion exchange matrix that was used for binding and enriching EVs saves working steps and materials. Furthermore, the use of chaotropic salts can be avoided. Depending on the anion exchange particles used, the bound extracellular RNA can then be eluted using a low salt, moderate pH elution buffer. Such elution buffer is compatible with many core downstream applications and does not require a further clean-up. This again saves handling steps and resources.

According to the present disclosure, the bound extracellular vesicles are lysed in the presence of at least one detergent which lyses the EVs and allows under the acidic lysis conditions the direct binding of the released RNA to the anion exchange particles. It is highly advantageous that the used detergent does not substantially inhibit binding of the released vesicular RNA to the anion exchange particles. The detergent used may be a non-ionic or anionic detergent.

To further improve the lysing step and remove contaminating proteins, a protease treatment can be included in the lysing step, and an additional wash step can be performed to eliminate EV components such as lipids, membrane proteins, etc. As a result, all kinds of proteins may be digested, including proteins that may bind to anion exchange particles by virtue of their net negative charge, or by interaction with other negatively charged biomolecules present.

Moreover, the method according to the present disclosure allows to use magnetic anion exchange particles as anion exchange particles. This avoids the use of expensive prior art anion exchange membranes that are commonly used for enriching EVs. Furthermore, the use of magnetic anion exchange particles renders the methods according to the present invention automatable. Thus, according to an advantageous embodiment, the solid phase in the binding step (aa) is provided by particles, such as magnetic particles. This allows to perform the methods of the present invention in an automated or semi-automated manner.

The anion exchange particles used preferably comprise anion exchange groups that allow for efficient binding of the released vesicular RNA while at the same time allow an efficient elution of bound RNA by using a moderate pH. The bound extracellular RNA can be eluted using high salt, or preferably using low salt buffers at moderate pH, such as 7-9, e.g. 8 to 9 or 8.5 to 9. The enriched extracellular RNA can in such case be directly used for downstream applications.

According to a first aspect, a method for enriching extracellular nucleic acids, such as preferably extracellular RNA from a sample comprising extracellular vesicles is provided, the method comprising the following steps:

(aa) preparing an acidic binding mixture comprising the sample and an anion exchange solid phase and binding extracellular vesicles to the anion exchange solid phase;

(bb) separating the anion exchange solid phase comprising the bound extracellular vesicles from the binding mixture;

(cc) lysing the bound extracellular vesicles in the presence of at least one detergent and binding released vesicular nucleic acids, such as preferably RNA, to the anion exchange solid phase;

(dd) separating the anion exchange solid phase with the bound nucleic acids from the lysate.

As is disclosed herein, the extracellular nucleic acids are preferably extracellular RNA which is the dominant nucleic acid in EVs. Moreover, while different formats of solid phases may be used (including filters and membranes), it is preferred to use anion exchange particles in the present methods. This is preferred because anion exchange particles, such as magnetic particles are easy to process and better suitable for automation. Therefore, the disclosure presented herein is presented with a focus on anion exchange particles. The skilled person will appreciate though that the disclosure also applies in a broader context and the use of other solid phase types.

According to a sub-aspect of the method according to the first aspect, a method for enriching extracellular RNA from a sample comprising extracellular vesicles is provided, the method comprising the following steps:
(aa) preparing an acidic binding mixture comprising the sample and anion exchange particles and binding extracellular vesicles to the anion exchange particles;
(bb) separating the anion exchange particles comprising the bound extracellular vesicles from the binding mixture;
(cc) lysing the bound extracellular vesicles in the presence of at least one detergent and binding released RNA to the anion exchange particles;
(dd) separating the anion exchange particles with the bound RNA from the lysate.

According to a second aspect, a kit for performing the method according to the first aspect is provided, comprising:

(a) anion exchange particles,

(b) an acidic reagent, preferably a buffer;

(c) an acidic lysis reagent, preferably a buffer which is different from the acidic reagent
(b) and comprises a detergent;

(d) optionally, one or more wash solutions; and

(e) optionally, one or more elution solutions.

The term “reagent” is used herein in a broad sense and covers the use of solutions as well as buffers. Other objects, features, advantages and aspects of the present application will become apparent to those skilled in the art from the following description and appended claims. It should be understood, however, that the following description, appended claims, and specific examples, while indicating preferred embodiments of the application, are given by way of illustration only.

### BRIEF DESCRIPTION OF THE FIGURES

FIG. 1A: Shows results of Example 2, here recovery of vesicular mRNA (EEF2). Shown are the Ct-values, wherein a lower Ct value indicates a better recovery.

FIG. 1B: Shows results of Example 2, here recovery of vesicular miRNA (let-7a).

FIG. 1C: Shows results of Example 2, here recovery of non-vesicular miRNA (miR-122) which may be co-isolated with the vesicular RNA.

FIG. 2: Shows results of Example 3, here RNA cleanup and elution at different conditions using four different anion exchange particle types (carrying PEI (AxpH), poly-histidine (p-His), oligo-histidine (10-His) or histamine, as indicated in the graph).

### DETAILED DESCRIPTION

As is demonstrated by the examples, extracellular RNA can be enriched and thus isolated from a sample comprising extracellular vesicles. The term enrichment is used in a broad sense and inter alia covers the isolation and purification of the target analyte, here extracellular RNA comprising vesicular RNA. The workflows described herein enable a highly simplified enrichment and analysis of extracellular RNA, in particular comprised in extracellular vesicles.

“Extracellular DNA” and “extracellular RNA” as used herein, in particular refers to DNA and RNA, respectively, that is not contained in cells but is comprised in the extracellular fraction of the biological sample, such as a (cell-containing) bodily fluid sample. Generally, extracellular nucleic acids are also often referred to as cell-free nucleic acids, such as cell-free DNA (“cfDNA”) and cell-free RNA (“cfRNA”). These terms are used as synonyms herein. Cell-free nucleic acids obtained from a circulating bodily fluid (such as blood) are also referred to as circulating cell-free nucleic acids, e.g. ccfDNA or ccfRNA. Extracellular nucleic acids may be enriched from a cell-depleted or cell-free fraction that may be obtained from a cell-containing bodily fluid (e.g. blood plasma or serum, preferably plasma). Extracellular RNA may be enriched from a sample, from which non-vesicular extracellular DNA has been removed in a previous step, such that the sample comprises less non-vesicular extracellular DNA than before. In particular, a cell-depleted or cell-free biological sample comprising extracellular vesicles may be subjected to a DNA depletion step, in particular by binding DNA, such as extracellular DNA, to particles comprising anion exchange groups, and separating the bound DNA from the binding mixture, whereby a DNA depleted sample comprising extracellular vesicles is provided that is subjected to step (aa) of the method according to the present disclosure for binding extracellular vesicles to the anion exchange particles. Such an extracellular DNA-depleted sample comprising extracellular vesicles can be used as a sample for the method of the present disclosure.

According to a particularly preferred embodiment, extracellular RNA is enriched using the method and kit according to the first and second aspect of the present disclosure. However, the method and kit may not be limited to the enrichment of extracellular RNA. It may also be suitable for isolating extracellular DNA that is comprised in extracellular vesicles. Vesicular DNA may be present in the plasma of cancer patients only (or perhaps other diseases, as well) and therefore, can represent an advantageous target that can be enriched using the method and kit according to the present disclosure. In particular, extracellular vesicles have also been shown to contain genomic DNA fragments from their cells of origin. The method and kit described in the present disclosure can be also used to separate such vesicular DNA (also from non-vesicular cell-free DNA as disclosed herein). Accordingly, the present method and kit may also be provided for enriching extracellular nucleic acids from a sample comprising extracellular vesicles, in particular for enriching extracellular RNA and/or DNA, which is comprised in extracellular vesicles.

Examples of typical extracellular nucleic acids that are found in the cell-free fraction of body fluids include but are not limited to mammalian extracellular nucleic acids such as e.g. extracellular tumor-associated or tumor-derived DNA and/or RNA, other extracellular disease-related DNA and/or RNA, epigenetically modified DNA, fetal DNA and/or RNA, small interfering RNA such as e.g. miRNA and siRNA, and non-mammalian extracellular nucleic acids such as e.g. viral nucleic acids, pathogen nucleic acids released into the extracellular nucleic acid population e.g. from prokaryotes (e.g. bacteria), viruses, eukaryotic parasites or fungi. The extracellular nucleic acid population usually comprises certain amounts of intracellular nucleic acids that were released from damaged or dying cells.

The term “extracellular vesicle” (EV) or “extracellular vesicles” (EVs) as used herein in particular refers to any type of secreted vesicle of cellular origin. EVs may be broadly classified into exosomes, microvesicles (MVs) and apoptotic bodies. EVs such as exosomes and microvesicles are small vesicles secreted by cells. EVs have been found to circulate through many different body fluids including blood and urine which makes them easily accessible. Due to the resemblance of EVs composition with the parental cell, circulating EVs are a valuable source for biomarkers. Circulating EVs are likely composed of a mixture of exosomes and MVs. They contain nucleic acids, in particular mRNA, miRNA, other small RNAs protected from degradation by a lipid bilayer. The contents are accordingly specifically packaged, and represent mechanisms of local and distant cellular communications. They can transport RNA between cells. EVs such as exosomes are an abundant and diverse source of circulating biomarkers. The cell of origin may be a healthy cell or a cancer cell. The cell of origin may also be an otherwise disease-affected or affected cell, including a stress-affected cell. For instance, the cell may be affected by a neurodegenerative disease. Another example is a stressed cell, such as a cell that underwent ageing. A stressed cell may release more EVs (and moreover extracellular DNA). EVs such as exosomes are often actively secreted by cancer cells, especially dividing cancer cells. As part of the tumor microenvironment, EVs such as exosomes seem to play an important role in fibroblast growth, desmoplastic reactions but also initiation of epithelial-mesenchymal transition (EMT) and SC as well as therapy resistance building and initiation of metastases and therapy resistance. There is thus a high interest in analyzing EVs, respectively EV content such as vesicular RNA.

As disclosed herein, the method and kit according to the first and second aspects of the present disclosure, respectively, are based on the same core principle for binding EVs to anion exchange particles, in particular magnetic anion exchange particles, and lysing said EVs in the presence of at least one detergent, such that the released RNA is bound to the anion exchange particles.

### The Method According to the First Aspect

The method according to the first aspect has been disclosed in the summary of the invention above.

According to a preferred embodiment, in step (aa) the extracellular vesicles are bound to anion exchange particles, as well as in step (cc) the released RNA. Although anion exchange particles, in particular magnetic anion exchange particles, are preferred, the method and kit of the present disclosure is not limited to anion exchange particles. The core principle of the present invention can also be advantageously used in order to enrich RNA extracellular RNA from a sample comprising extracellular vesicles by preparing an acidic binding mixture comprising the sample and an anion exchange solid phase and binding extracellular vesicles to the anion exchange solid phase. An anion exchange solid phase may be selected from any solid phase known in the art for enriching analytes. In particular, a solid phase can be selected from a porous solid phase, such as a membrane or a column. The solid phase preferably comprises anion exchange groups as defined in the present disclosure to which is referred here. Rebinding of the released vesicular nucleic acids to an anion exchange membrane or filter may be achieved by reapplying the lysate.

According to a preferred embodiment of the method according to the first aspect, a method for enriching extracellular RNA from a sample comprising extracellular vesicles is provided, the method comprising the following steps:
(aa) preparing an acidic binding mixture comprising the sample and anion exchange particles and binding extracellular vesicles to the anion exchange particles;
(bb) separating the anion exchange particles comprising the bound extracellular vesicles from the binding mixture;
(cc) lysing the bound extracellular vesicles in the presence of at least one detergent and binding released RNA to the anion exchange particles;
(dd) separating the anion exchange particles with the bound RNA from the lysate.

The method according to the first aspect allows to enrich extracellular RNA from a sample comprising extracellular vesicles by binding extracellular vesicles to the anion exchange particles, followed by separating the particles and lysing the bound extracellular vesicles in the presence of at least one detergent, while allowing binding of the released RNA to the anion exchange particles, as is demonstrated by the examples. The method according to the first aspect, allows to advantageously isolate extracellular RNA which is in particular comprised in extracellular vesicles without the need to a complicated and time-consuming clean-up. By lysing the extracellular vesicles bound to the anion exchange particles and binding the released RNA, the vesicular RNA can be directly enriched. Moreover, by selection of the anion exchange particles, in particular the type of anion exchange groups, the extracellular RNA can be bound such to the anion exchange particles, that these can be easily eluted from the particles by an elution buffer comprising a high or preferably a low salt concentration. Hence, enriched RNA can be yielded which can be directly used for analysis without the necessity of a subsequent workup protocol for removing elution buffer compound, such as phenol, e.g. from QIAzol.

According to a preferred embodiment, the method comprises the further steps of
(ee) optionally washing the bound RNA; and
(ff) eluting the bound RNA from the anion exchange particles.

Further preferred embodiments of optional step (ee) and step (ff) are disclosed further below.

Step (aa)

Step (aa) comprises preparing an acidic binding mixture comprising the sample (comprising extracellular vesicles) and anion exchange particles and binding extracellular vesicles to the anion exchange particles.

According to step (aa), an acidic binding mixture is prepared that comprises the sample and anion exchange particles and the extracellular vesicles are bound to the anion exchange particles. Thus, the acidic binding mixture provides advantageously binding conditions for binding the extracellular vesicles to the anion exchange particles. Furthermore, apart from the extracellular vesicles, further extracellular RNA that is comprised in the sample may be bound to the anion exchange particles. For instance, non-vesicular cell-free RNA (cfRNA) or circulating cell-free RNA (ccfRNA) may be comprised in the sample, which can also bind to the anion exchange particles together with the extracellular vesicles. According to one embodiment, extracellular DNA predominantly does not bind to the solid phase in step (aa). This can be achieved in particular by providing a sample comprising extracellular vesicles that is already extracellular DNA-depleted. For instance, a sample can be provided, wherein the extracellular DNA was removed prior to step (aa), e.g. by binding the extracellular DNA to a solid phase comprising anion exchange particles. Suitable examples are described herein.

According to a preferred embodiment, the anion exchange particles are magnetic anion exchange particles. Using magnetic anion exchange particles has the advantage that the particles can be easily separated from the remaining mixture by providing a magnetic field, e.g. in form of a magnet. As the provision of a magnetic field can be easily achieved by an automated platform, using magnetic anion exchange particles has the advantage that the method can be fully or partially performed in an automated manner.

Further preferred embodiments of step (aa) are disclosed further below.

Step (bb)

Step (bb) comprises separating the anion exchange particles (or other solid phase) comprising the bound extracellular vesicles from the binding mixture.

According to a preferred embodiment, step (bb) comprises washing the separated anion exchange particles with the bound extracellular vesicles. Preferably, the extracellular vesicles are bound to the anion exchange particles when washing is performed in step (bb). Washing may be performed with any suitable wash solution, which is applied in the art. According to one embodiment, for washing the components of the acidic reagent are used at a concentration as provided in the acidic binding mixture of step (aa). Disclosures related to the acidic reagent and the acidic binding mixture can be found below and it is here referred thereto. Washing the separated anion exchange particles with the bound extracellular vesicles can further reduce the amounts of contaminants such as proteins comprised in the sample.

In separation step (bb) (and likewise separation step (dd)) the anion exchange particles may be separated by centrifugation, sedimentation or magnetic separation. Preferably, magnetic anion exchange particles are used that may be separated by aid of a magnetic field. Magnetic anion exchange particles are particularly preferred, as these can be easily separated by a magnetic field from the remaining solution, allowing a sample transfer to existing automated platforms for performing the separation step (bb) and/or (dd) in an automated manner. Further ways of separating anion exchange particles can be readily found in the field and can also be applied in scope of step (bb) and/or step (dd) according to the present disclosure.

If a different type of solid phase is used, a different separation principle may be applied such as centrifugation, vacuum application and the like.

Step (cc)

Step (cc) comprises lysing the bound extracellular vesicles in the presence of at least one detergent and binding released RNA to the anion exchange particles.

Step (cc) of the present disclosure advantageously allows to lyse bound extracellular vesicles to release the comprised RNA. The released RNA then directly binds to the anion exchange particles. Therefore, no different binding solid phase needs to be provided but the anion exchange particles present in the lysis mixture which were used in step (aa) to bind the

EVs are directly utilized to bind the released RNA. Therefore, step (cc) is a simple and fast way of rendering the extracellular RNA which is comprised in extracellular vesicles into a form that allows a direct isolation by binding to the present anion exchange particles. Advantageously, the detergent that is used in step (cc) does not substantially inhibit binding of the released vesicular RNA to the anion exchange particles. Therefore, the detergent does not need to be removed in order to enable binding of the released RNA to the anion exchange particles. In step (cc), lysis conditions are provided that allow direct binding of the released RNA to the anion exchange particles.

Moreover, by lysing the extracellular vesicles and binding the released RNA to the anion exchange particles, the bound product can be directly used, e.g. for downstream applications, such as analysis. According to one embodiment, the extracellular RNA is bound to the anion exchange particles, wherein the anion exchange particles are configured such that an elution is possible using a mild, low salt concentration elution buffer.

According to a preferred embodiment, step (cc) comprises preparing a lysis mixture by contacting the separated anion exchange particles comprising the bound extracellular vesicles with an acidic lysis reagent which comprises the at least one detergent suitable to lyse extracellular vesicles so that vesicular RNA is released into the lysate.

According to a preferred embodiment, the detergent is used in a concentration so that lysis of the bound extracellular vesicles and release of vesicular RNA is achieved. Advantageously the detergent does not or not substantially negatively interfere with the binding of the released RNA to the anion exchange particles. Hence, the detergent is provided at a concentration sufficient for lysis and release of RNA but allowing for binding of the released RNA to the anion exchange particles. Detergent-based lysis of extracellular vesicles is also disclosed in the art (see e.g. Osteikoetxea et al Org Biomol Chem 2015 Oct. 14; 13 (38):9775). The concentration of the detergent in the lysis- and rebinding step can be chosen in accordance with the choice of the detergent to achieve efficient lysis of the extracellular vesicles and rebinding.

Concentrations or concentration ranges indicated in percentage values as used herein are in particular given as percentage weight per volume (w/v) for solid compounds, solid substances or solid compositions in a liquid composition, and as percentage volume per volume (v/v) for liquid compounds, liquid substances or liquid compositions in a liquid composition. For instance, SDS may be added in w/v percentages and Triton X-100 or Tween 20 as v/v percentages. In further embodiments, the indicated concentrations are intended to mean w/v for all embodiments, i.e. liquid and solid compounds.

According to one embodiment, in step (cc) the lysis mixture comprising the anion exchange particles comprises the detergent in a concentration of at least 0.1%, such as at least 0.2%, such as at least 0.3%, such as at least 0.4%, at least 0.5%, at least 0.75% or at least 1%.

According to a preferred embodiment, in step (cc) the lysis mixture comprising the anion exchange particles comprises the detergent in a concentration of at least 1.25%, such as at least 1.5%, at least 1.75% or at least 2%.

According to one embodiment, in step (cc) the lysis mixture comprising the anion exchange particles comprises the detergent in a concentration of 15% or less, 10% or less, 7% or less or 5% or less.

According to a preferred embodiment, step (cc) comprises preparing a lysis mixture by contacting the separated anion exchange particles comprising the bound extracellular vesicles with a lysis reagent which comprises the detergent, wherein the detergent is comprised in the lysis mixture in a concentration in a range of 0.1 to 15%, such as 0.5 to 10%, 0.75% to 7% or 1% to 5%.

According to a preferred embodiment, the detergent used in step (cc) for lysing the extracellular vesicles is not a cationic detergent. Without being bound to theory, it is believed that a cationic detergent can interfere with the binding of the extracellular vesicles to the anion exchange particles.

According to a preferred embodiment, the at least one detergent used in step (cc) for lysing the extracellular vesicles is selected from a non-ionic surfactant and an anionic detergent. Without being bound to theory, non-ionic surfactants and anionic surfactants are believed to not interfere or not substantially interfere with the binding of the released RNA and the anion exchange particles, when being provided in the lysis mixture. Thus, the detergent according to the present invention advantageously allows for binding of the released RNA to the anion exchange particles in the lysis mixture. Suitable embodiments are disclosed herein and in the working examples.

According to one embodiment, the detergent used in step (cc) for lysing the extracellular vesicles is a non-ionic detergent, preferably a polyoxyethylene-based non-ionic detergent. Such non-ionic detergent can be selected from the group consisting of (i) polyoxyethylene fatty alcohol ethers, (ii) polyoxyethylene alkylphenyl ethers, (iii) polyoxyethylene-polyoxypropylene block copolymers, (iv) polyoxyethylene fatty acid esters, (v) ethoxylated propoxylated alcohols, (vi) steroidglycoside-based non-ionic detergents and (vii) sorbitan fatty acid esters. Optionally, the non-ionic detergent has at least one of the following characteristics:
(i) it is a polyoxyethylene fatty alcohol ether, optionally comprising a fatty alcohol component having 4 to 28 carbon atoms, and a polyoxyethylene component having 2 to 150 (CH2CH2O) units, optionally selected from a polyoxyethylene lauryl ether, such as polyoxyethylene(4) lauryl ether (e.g. Brij® 30) or polyoxyethylene(23) lauryl ether (e.g. Brij® 35), a polyoxyethylene cetyl ether, such as polyoxyethylene(10) cetyl ether (e.g. Brij® 56) or polyoxyethylene(20) cetyl ether (e.g. Brij® 58), a polyoxyethylene stearyl ether, such as polyoxyethylene(2) stearyl ether (e.g. Brij® 72) or a polyoxyethylene(20) stearyl ether (e.g. Brij® 78), and a polyoxyethylene oleyl ether, such as polyoxyethylene(20) oleyl ether (e.g. Brij® 98);
(ii) it is a polyoxyethylene alkylphenyl ethers, optionally a polyoxyethylene octylphenyl ethers or polyoxyethylene nonylphenyl ethers, optionally branched, optionally selected from a polyoxyethylene p-isooctylphenyl ether (e.g. Triton™ X-100), a polyoxyethylene tert-octylphenyl ether (e.g. Triton™ X-114), a polyoxyethylene (40) isooctylphenyl ether (e.g. Triton™ X-450), a octylphenoxy poly(ethyleneoxy)ethanol (e.g. Igepal® CA-630) or a 4-Nonylphenyl-polyethylene glycol;
(iii) it is a polyoxyethylene-polyoxypropylene block copolymer, such as a poloxamer;
(iv) it is a polyoxyethylene fatty acid ester, such as polyoxyethylene sorbitan monolaurate (Tween® 20), polyoxyethylene sorbitan monooleate (Tween® 80);
(v) it is a ethoxylated propoxylated alcohol, such as seed oil alcohol ethoxylates, in particular seed oil alcohol ethoxylates 4 EO (ECOSURF™ SA-4), seed oil alcohol ethoxylate 7 EO (ECOSURF™ SA-7) or seed oil alcohol ethoxylate 9 EO (ECOSURF™ SA-9);
(vi) it is a steroidglycoside-based non-ionic detergent, such as Digitonin; and/or
(vii) it is a sorbitan fatty acid ester, such as sorbitan monolaurate (e.g. Span® 20), sorbitan monostearate (e.g. Span® 60) or sorbitan monooleate (e.g. Span® 80).

Nonionic detergents preferably include polyoxyethylene detergents, especially polyoxyethylene alkyl ethers, wherein the alkyl moiety is preferably a straight or branched alkyl group of 4 to 28, preferably 8 to 24, more preferably 12 to 20 carbon atoms and the polyoxyethylene moiety at least 2, preferably at least 4, more preferably having from 6 to 24 ethylene units, polyoxyethylene alkylaryl ethers in which a phenyl group preferably para-substituted with an alkyl group is coupled to the polyoxyethylene group, and Triton X series detergents.

According to one embodiment, the detergent used in step (cc) for lysing the extracellular vesicles is an anionic detergent, optionally a sulfate or sulfonate of a fatty alcohol. A suitable anion detergent can be selected from the group consisting of
(i) a sulfate or sulfonate of a fatty alcohol, such as sodium dodecyl sulfate, sodium dodecyl sulfonate or dodecylbenzenesulfonic acid;
(ii) a bile-acid based detergent, such as deoxycholate, in particular sodium deoxycholate, or sodium cholate, and
(iii) a sarcosine-based detergent, such as sarkosyl or N-lauroylsarcosine.

Optionally, the anionic detergent is selected from the group of sodium dodecyl sulfate, sodium dodecyl sulfonate, dodecylbenzenesulfonic acid, N-lauroylsarcosine and sodium cholate, and wherein the anionic detergent optionally is sodium dodecyl sulfate.

Preferred anionic detergents are sulfates, sulfonates, phosphates and carboxylic acids, preferably as salt, for example as alkali metal salt or alkaline earth metal salts, e.g. as sodium, lithium or potassium salt, or as the free acid. Anionic detergents may in particular be sulfates of fatty alcohols, in particular having an unbranched or branched alkyl chain of 4 to 28 carbon atoms, preferably 8 to 18 carbon atoms, or alkylarylsulfonates, in particular linear alkylbenzenesulfonates. Specific examples of anionic detergents are sodium dodecylsulfate (SDS), lithium dodecylsulfate, sodium octylsulfate, sodium dodecylsulfonate, sodium decylsulfonate, sodium octylsulfonate, dodecylbenzenesulfonic acid (DDBSA), N-lauroylsarcosine, sodium cholate and sodium deoxycholate.

According to a particular embodiment, the detergent used for extracellular vesicle lysis in step (cc) is selected from the group of Triton X-100, sodium dodecyl sulfate, deoxacholate, sarcosyl and/or Ecosurf SA-9.

According to a preferred embodiment, the acidic lysis reagent of step (cc) comprises
(i) the at least one detergent, optionally in a concentration as defined above for the lysis mixture; and
(ii) a buffering agent.

In particular, the concentration of the at least one detergent in the lysis reagent of step (cc) may be selected from the range of 0.1 to 15%, such as 0.5 to 10%, 0.75% to 7% or 1% to 5%.

According to one embodiment, the acidic lysis reagent has an acidic pH that promotes binding of the released vesicular RNA to the anion exchange groups of the magnetic particles. By providing a pH suitable for binding the released RNA in step (cc), no further components need to be added in order to bind the released RNA to the anion exchange particles. If desired, however, further components can be added to the lysis mixture of step (cc) to enhance binding of the released RNA to the anion exchange groups.

According to a preferred embodiment, the acidic lysis reagent has a pH in the range of 2.5 to 5.5, such as 2.7 to 5.3, 3 to 5 or 3 to 4.7. Particular pH values of the acidic lysis reagent can be found by in the examples of the present invention. According to one embodiment, a pH in the range of 3.5 to 4.5 is provided, such as 4.

According to one embodiment the pH of the acidic lysis reagent is ≤5, optionally ≤4.7, ≤4.5 or ≤4.3.

According to a preferred embodiment, the acidic lysis reagent used in step (cc) comprises a carboxylic acid based buffering agent, optionally acetate. Other buffering agents may also be used for the acidic lysis reagent in step (cc), in particular acidic buffering agents known in the art. The buffering agent may be present in a concentration of ≤500 mM, such as ≤450 mM, ≤400 mM, ≤350 mM, preferably ≤300 mM or ≤250 mM in the acidic lysis reagent in step (cc).

According to a preferred embodiment, the acidic lysis reagent establishes conditions that allow direct binding of the released vesicular RNA to the anion exchange particles.

According to one embodiment, the total salt concentration in the acidic lysis reagent is 1M or less, preferably 0.75M or less, 0.5M or less or 370 mM or less. Further salt concentration may be found suitable, as long as the extracellular RNA, in particular the released vesicular RNA binds to the anion exchange particles.

According to one embodiment, total salt concentration in the acidic lysis reagent is 350 mM or less, such as 325 mM or less, 300 mM or less or 275 mM or less. It may be particularly suitable to provide a total salt concentration in the acidic lysis reagent that does not interfere but preferentially enhance the binding of the released RNA to the anion exchange particles.

According to one embodiment, the acidic lysis reagent used in step (cc) does not comprise a chaotropic salt and/or an organic solvent. An acidic lysis reagent in step (cc) that does not comprise a chaotropic salt and/or an organic solvent may be understood such the acidic lysis reagent does not comprise such compounds at all or at a non-effective amount. Therefore, these compounds advantageously do not interfere with the binding of the released vesicular RNA to the anion exchange particles.

Protease

According to a preferred embodiment, step (cc) comprises adding a protease. The protease can advantageously assist lysis and therefore, improve the yield of the vesicular RNA. Without being bound to theory, a protease, such as proteinase K, is believed to inactivate degradative enzymes that may be present in the lysis mixture of step (cc). Moreover, contaminants may be removed such as protein which bound to the anion exchange groups or solid phase in step (aa). Therefore, degradation of the extracellular RNA present in the lysis mixture is reduced.

According to a preferred embodiment, the protease is a proteinase, preferably proteinase K.

According to one embodiment, under the conditions established by the acidic lysis reagent agent in step (cc), vesicular RNA released form the lysed extracellular vesicles binds to the anion exchange particles present in the lysis mixture. Such binding is advantageously not negatively affected by compounds of the acidic lysis reagent but preferably enhanced, e.g. by provision of a suitable pH and buffering agent.

According to one embodiment, step (cc) comprises contacting the separated anion exchange particles comprising the bound extracellular vesicles with the acidic lysis reagent and wherein no further reagents are added to establish the EV lysis and vesicular RNA binding conditions in step (cc). Such an embodiment is advantageous, as further processing steps are avoided, as for example, no further anion exchange particles need to be added or further buffering or acidic reagent.

According to one embodiment, step (cc) comprises incubating the lysis mixture to allow lysis of extracellular vesicles and direct binding of the released vesicular RNA to the anion exchange particles. According to a particular embodiment, incubation is performed at room temperature or above. For instance, it may be found suitable to increase the temperature such that an added protease is more active, e.g. by heating to a temperature of 30° C. or more, 35° C. or more, 40° C. or more or 45° C. or more. Suitable temperatures may be readily found in the art by the skilled person.

Step (dd)

Step (dd) comprises separating the anion exchange particles with the bound RNA from the lysate.

According to a preferred embodiment, the anion exchange particles that are separated in step (dd) from the lysate comprise bound thereto extracellular RNA which comprises vesicular RNA and optionally non-vesicular RNA. Binding of non-vesicular RNA may have been achieved in the binding mixture of step (aa) by binding extracellular non-vesicular RNA to the anion exchange particles present in the sample.

According to a particular embodiment, the bound RNA comprises non-vesicular RNA that was bound together with the extracellular vesicles in step (aa) to the anion exchange particles.

Ways and techniques for separating anion exchange particles have been described above in conjunction with step (bb) and it is here referred thereto. According to a particular embodiment, magnetic anion exchange particles are used, facilitating separation by magnetic forces, e.g. a magnet.

Step (ee)

According to a preferred embodiment, the method further comprises optional step (ee) comprising washing the bound RNA.

According to one embodiment, step (ee) comprising washing the bound RNA is mandatory. According to one embodiment, the method according to the first aspect comprises performing one or more wash steps (ee). At the end of a wash step, it may be preferred to separate the anion exchange particles from the wash solution to remove the wash solution. In case the washing step is performed multiple times it may be preferred to separate the anion exchange particles after every washing step.

Suitable wash solution may be found in the art and are known by the skilled person. According to a particular embodiment, the wash solution may correspond to the acidic lysis reagent as defined above. Optionally, the wash solution corresponds to the acidic lysis reagent as defined above without comprising a detergent.

Step (ff)

According to a preferred embodiment, the method further comprises step (ff) comprising eluting the bound RNA from the anion exchange particles.

The method according to the first aspect allows in a preferred embodiment to elute the extracellular RNA such that the extracellular RNA does not need to be purified in a complex and time-consuming subsequent workup. Advantageously, the bound extracellular RNA may be eluted from the anion exchange particles, such as magnetic anion exchange particles, by providing an elution buffer that allows for the extracellular RNA to be directly analyzed.

According to a preferred embodiment, the elution step (ff) is performed using one or more elution solutions. According to a preferred embodiment, the elution solution has a basic pH, preferably of at least 8.0, at least 8.3 or at least 8.5 and wherein preferably, the pH of the elution solution is ≤9 or <9. According to one embodiment, the elution solution comprises a buffering agent, optionally selected from TRIS, HEPES, HPPS or an ammonia buffer, preferably TRIS.

According to one embodiment, elution comprises a heating step. The heating step may improve the elution and/or allow using an elution solution that comprises less salt, which can advantageously allow to obtain enriched extracellular RNA, which can be directly used for analysis, avoiding subsequent clean-up, e.g. by removal of salts.

According to one embodiment, the elution solution as described above is used in combination with anion exchange particles comprising anion exchange groups that release the bound RNA at the elution conditions provided by the elution solution. This is particularly advantageous, as harsh elution conditions are avoided, which necessitate a subsequent clean-up, e.g. by removal of salt or organic compounds. Accordingly, the anion exchange groups may comprise per anion exchange group at least one amino group, optionally 1 to 20 or 1 to 15 amino groups. Preferably, the amino group is part of an imidazole ring. According to a particular embodiment, the anion exchange groups comprise histidine or histamine. According to a preferred embodiment, the anion exchange groups of the particles are selected from (i) oligo-histidine, wherein the number of histidine monomers is in the range of 4 to 18, such as 5 to 16, 6 to 14, 7 to 13 or preferably 8 to 12, and (ii) a histamine group, optionally wherein the anion exchange groups comprise 1 histamine group per anion exchange group.

According to one embodiment, the total salt concentration in the elution solution is 500 mM or less, such as 250 mM or less, 200 mM or less, 150 mM or less or 100 mM or less, optionally 50 mM or less. According to a particular embodiment, the elution solution comprises a salt concentration selected from 5 to 250 mM. For instance, the elution solution may comprise 200 mM or 10 mM salt, in particular 200 mM or 10 mM Tris.

According to one embodiment, the total salt concentration in the elution solution is at least 500 mM, such as at least 750 mM, at least 1M or at least 1.2M. Such a salt concentration may be advantageous when providing anion exchange particles that bind the extracellular RNA stronger. For instance, polyethyleneimine-based or poly-histidine-based anion exchange particles, which strongly bind the extracellular RNA may require an elution solution comprising said salt concentration.

According to one embodiment, the elution solution is an extraction reagent, optionally wherein the elution solution comprises phenol and/or comprises a chaotropic salt, optionally selected from guanidinium alts, thiocyanate salts, iodide salts, perchlorate salts, trichloroacetate salts and trifluroacetate salts. Optionally such an elution solution has a pH of at least 7.5 or at least 8.

### The Kit According to the Second Aspect

According to a second aspect, a kit for performing the method according to the first aspect is provided, comprising:
(a) anion exchange particles,
(b) an acidic reagent;
(c) an acidic lysis reagent which is different from the acidic reagent (b) and comprises a detergent;
(d) optionally, one or more wash solutions; and
(e) optionally, one or more elution solutions.

The kit can be used in order to perform the method according to the first aspect. The advantages were described above, including the used reagents and the solid phase. Including an acidic lysis reagent (c), which allows to lyse extracellular vesicles bound to the anion exchange particles and bind the released RNA to the anion exchange particles. Therefore, not only lysing but also binding conditions are established at the same time. The enriched RNA are of high quality and purity. The kit and corresponding method thereby becomes easily usable and can avoid subsequent complicated and time-consuming clean-up workflows, as the extracellular RNA can be directly enriched using the anion exchange particles provided in step (aa) by kit component (a).

The anion exchange particles that may be preferably used in the kit according to the present disclosure are described throughout the present disclosure and it is here referred thereto. According to a preferred embodiment, the anion exchange particles are magnetic anion exchange particles. Particular anion exchange groups are described below. According to one embodiment, the anion exchange groups comprise at least one amino group. According to a particular embodiment, the anion exchange groups of the particles are selected from (i) polyethyleneimine; (ii) polyhistidine, wherein the number of histidine monomers is at least 30; (iii) oligo-histidine, wherein the number of histidine monomers is in the range of 4 to 18, such as 5 to 16, 6 to 14, 7 to 13 or preferably 8 to 12, and (iv) histamine.

According to a preferred embodiment, the acidic reagent of the kit is an acidic reagent as described below to which we refer here. According to one embodiment, the acidic reagent comprises buffering agent and provides a pH that allows for binding the extracellular vesicles to the anion exchange particles. According to one embodiment, the pH of the acidic reagent is in the range of 2 to 5, such as 2.5 to 5, preferably 3 to 5, more preferably 3 to 4.5. Moreover, the acidic reagent may comprise a carboxylic acid based buffering agent, such as an acetate buffer, optionally provided by a sodium acetate/acetic acid buffer.

The acidic lysis reagent comprising a detergent has been described above and it is here referred thereto. According to a particular embodiment, the detergent is not a cationic detergent. Accordingly, a preferred detergent is selected from a non-ionic surfactant and an anionic detergent. The detergent is advantageously provided at a concentration sufficient to lyse the extracellular vesicles in the lysis mixture. In particular, the detergent may have a concentration in the acidic lysis reagent of at least 0.1%, such as at least 0.2%, such as at least 0.3%, such as at least 0.4%, at least 0.5%, at least 0.75% or at least 1%, or at least 1.25%, such as at least 1.5%, at least 1.75% or at least 2%. The concentration of the detergent in the lysis reagent of step (cc) may be selected from the range of 0.1 to 15%, such as 0.5 to 10%, 0.75% to 7% or 1% to 5%.

The acidic lysis reagent may be selected from an acidic lysis reagent as described throughout the present disclosure. An acidic lysis reagent may preferably comprise a buffering agent as defined above and provide a pH in the lysis mixture allowing for binding the released RNA to the anion exchange particles. Accordingly, the acidic lysis reagent establishes conditions that allow direct binding of the released vesicular RNA to the anion exchange particles.

According to one embodiment, the kit comprises an elution solution. Suitable elution solution have been described above and it is here referred thereto. It may be particularly suitable to provide an elution solution comprising a low salt concentration such as a total salt concentration in the elution solution of 500 mM or less, such as 250 mM or less, 200 mM or less, 150 mM or less or 100 mM or less, optionally 50 mM or less.

The kit may comprise further components, such as one or more washing solution and/or a protease, such as proteinase K, which are described in the present disclosure which also applied here. Further features which can be used in conjunction with the method according to the first aspect may also be integrated into the kit according to the second aspect.

### Specific Embodiments

Further embodiments of the present invention are described in the following.

The Sample

According to one embodiment, the sample comprising extracellular vesicles is or is derived from a body fluid. The sample is preferably a sample obtained from a body fluid by removing cells. The sample may be a cell-free or cell-depleted body fluid sample. According to one embodiment, the cell-free or cell-depleted body fluid sample is or is derived from the following samples by removing cells: whole blood, plasma, serum, lymphatic fluid, urine, liquor, cerebrospinal fluid, synovial fluid, interstitial fluid, ascites, milk, bronchial lavage, saliva, amniotic fluid, semen/seminal fluid, body secretions, nasal secretions, vaginal secretions, wound secretions and excretions. According to a particular embodiment, the sample is selected from plasma, serum and urine, wherein urine is preferably cell-depleted or cell-free urine.

According to a particular embodiment, the method comprises removing cells from a body fluid sample, whereby a cell-depleted body fluid sample is provided as sample comprising extracellular vesicles, wherein said sample is contacted in step (aa) with the anion exchange particles and preferably an acidic reagent to prepare the acidic binding mixture.

According to one embodiment, the sample is a cell culture supernatant comprising extracellular vesicles. According to one embodiment, the sample comprising extracellular vesicles is a plant extract such as a fruit extract.

DNA Depletion Step

According to one embodiment, prior to step (aa) a cell-depleted or cell-free biological sample comprising extracellular vesicles is subjected to a DNA depletion step, in particular by binding DNA, such as extracellular DNA, to a particles comprising anion exchange groups, and separating the bound DNA from the binding mixture, whereby a DNA depleted sample comprising extracellular vesicles is provided that is subjected to step (aa) of the method according to any one of the preceding claims for binding extracellular vesicles to the anion exchange particles.

According to a particular embodiment, the method comprises
(a) preparing a binding mixture comprising
   a biological sample comprising extracellular vesicles, wherein preferably the biological sample is a cell-depleted or cell-free body fluid sample,
   anion exchange particles,
   an acidic reagent comprising a buffering agent,
   and binding extracellular DNA to the particles;
(b) separating the particles with the bound extracellular DNA from the binding mixture, wherein the remaining binding mixture provides a sample comprising extracellular vesicles; and
(c) enriching extracellular vesicles from the remaining binding mixture that provides a sample comprising extracellular vesicles using the method comprising steps (aa) to (dd) according to items 1 to 76 described in further detail below and as described in conjunction with the method according to the first aspect.

Such method is disclosed in PCT application PCT/EP2020/086576 filed by the applicant today and the corresponding priority application EP19216746.8, both herein incorporated by reference. As disclosed, this embodiment can be used to deplete non-vesicular DNA, e.g. DNA released by cell death, from a biological sample in advance of the EV binding step. Such release of non-vesicular DNA can occur e.g. during sample collection or cell culture. Binding of extracellular vesicles and other extracellular RNA to the solid phase comprising anion exchange groups in the extracellular DNA binding step (a) can be reduced or even eliminated by choice of the binding conditions and the anion exchange groups of the solid phase, in particular by adjusting the acidic pH of the used binding buffer. Binding of EVs to the anion exchange groups of the solid phase is more sensitive to pH changes compared to binding of cfDNA. Thus, while cfDNA shows a similar binding efficiency over a broader range of acidic pH, EVs bind less effectively at higher pHs. As disclosed in EP19216746.8 and PCT/EP2020/086576 this can be used to establish acidic binding conditions in the binding mixture in step (a), under which the extracellular DNA still binds with good yield to the anion exchange groups of the solid phase, while binding of EVs to the anion exchange groups of the solid phase used for cfDNA binding is already significantly reduced. According to one embodiment disclosed in EP19216746.8 and PCT/EP2020/086576, in extracellular DNA binding step (a) the pH of the binding mixture is in a range of 3.5 to 6, 3.7 to 5.5 or 4 to 5.2, optionally wherein the pH of the binding mixture is ≥4, ≥4.2 or ≥4.5. The acidic binding buffer that can be used in step (a) to establish in the binding mixture the conditions for binding extracellular DNA to the anion exchange particles may comprise a carboxylic acid based buffering agent. The buffering agent may comprise a buffer component selected from citrate, oxalate, formate, acetate, propionate, lactate and tartrate, in particular selected from citrate or oxalate, more preferably citrate. As disclosed in EP19216746.8 and PCT/EP2020/086576, the extracellular DNA may be bound using these acidic binding conditions e.g. to anion exchange particles, in particular magnetic anion exchange particles, which comprise anion exchange groups comprising a trialkylamine group or dialkylaminoalkyl group. Binding of extracellular vesicles to the anion exchange particles is, however, reduced under such binding conditions that are used in step (a). In step (b), the particles with the bound extracellular DNA are separated from the remaining binding mixture which comprises the unbound extracellular vesicles. Extracellular vesicles and optionally other extracellular RNA are thus predominantly contained in the remaining binding mixture that is provided in step (b). This remaining binding mixture from which the anion exchange particles with the bound extracellular DNA were removed provides a sample comprising extracellular vesicles. Extracellular vesicles are then enriched from the remaining binding mixture in step (c) using steps (aa) to (dd) of the method according to the first aspect disclosed herein. As disclosed herein, performing steps (aa) to (dd) provides nucleic acids released from the enriched extracellular vesicles bound to an anion exchange solid phase. The bound nucleic acids may then be optionally washed and eluted in steps (ee) and (ff) as disclosed herein. This provides the vesicular nucleic acids, such as vesicular RNA, in pure form.

This embodiment also allows to isolate cell-free DNA in a pure form, e.g. by optionally washing and eluting the bound extracellular DNA from the anion exchange particles that were separated in step (b). Such processing that can be performed prior to step (aa) of the method according to the first aspect of the present invention can advantageously also be used to deplete other undesired components prior to enriching the EVs, such as non-vesicular negatively charged proteins.

Step (aa)

Step (aa) of the method according to the first aspect is disclosed above. In the following disclosure, further embodiments of step (aa) are described, which can be advantageously used in order to establish conditions for binding the extracellular vesicles and optionally further extracellular (non-vesicular) RNA to the anion exchange particles.

pH of the Binding Mixture and Acidic Reagent

According to a preferred embodiment, the acidic binding mixture prepared in step (aa) has a pH in the range of 2 to 6, such as 2.5 to 5.5, preferably 3 to 5, more preferably 3 to 4.5. The provided pH advantageously allows to bind the extracellular vesicles to the anion exchange particles, in particular to magnetic anion exchange particles.

According to one embodiment, preparing the EV binding conditions in step (aa) comprises adding an acidic reagent, optionally wherein the pH of the acidic reagent is in the range of 2 to 5, such as 2.5 to 5, preferably 3 to 5, more preferably 3 to 4.5.

According to a preferred embodiment, the acidic reagent comprises a buffering agent, preferably a carboxylic acid based buffer. Accordingly, the carboxylic acid based may have a carboxylic acid based buffering agent comprising a carboxylic acid and a salt of said carboxylic acid, wherein preferably the carboxylic acid (i) comprises 1 to 3 carboxylic acid groups, (ii) is aliphatic, and/or (iii) is saturated. The carboxylic acid based buffering agent may comprise 1 to 3 carboxyl group, preferably 1 carboxyl group. According to a particular embodiment, the carboxylic acid based buffer is an acetate buffer, optionally provided by a sodium acetate/acetic acid buffer.

According to a preferred embodiment the EV binding mixture in step (aa) comprises the buffering agent from the acidic reagent in a concentration of 100 mM to 1M, preferably <1M, such as 200 mM to 700 mM, 300 mM to 600 mM or 350 mM to 550 mM, optionally wherein the buffering agent is acetate. Accordingly the buffering agent may be provided by a sodium acetate/acetic acid buffer.

According to a preferred embodiment, in step (aa) the pH of the binding mixture for binding extracellular vesicles to the anion exchange particles is lower than the pKa of the ionized form of the anion exchange groups of the particles, optionally wherein the pH is at least 1, at least 1.5, at least 2 or at least 2.5 unit(s) lower than the pKa. Suitable pKa values are described below.

According to one embodiment, in step (aa) the pH of the binding mixture corresponds to the pH of the acidic reagent that is added to adjust the binding conditions or deviates by ≤0.75 or preferably ≤0.5 pH units therefrom. The deviation may depend on the buffering strength and/or concentration of buffering agent provided by the acidic reagent.

According to a particularly preferred embodiment, magnetic anion exchange particles are used in step (aa). Magnetic anion exchange particles allow for performing the method according to the present disclosure in an automated or semi-automated manner.

According to one embodiment, the anion exchange particles comprise anion exchange groups at the surface of the particles. The anion exchange groups of the anion exchange particles may comprise anion exchange groups of the same or different types. According to one embodiment, the anion exchange groups are of the same type. According to one embodiment, the anion exchange groups are attached to the surface of the particles by covalent attachment, optionally using carbodiimide-based reactions, in particular by reacting carboxyl groups of the particles with amino groups comprised in the anion exchange groups. Suitable ways of attaching anion exchange groups to particles, in particular silica particles, such as magnetic silica particles, are well-known in the field. Moreover, suitable ways can be found in the example section of the present disclosure.

According to a preferred embodiment, the anion exchange groups comprise at least one ionizable group as functional group, wherein preferably the ionizable group is ionizable by protonation. Ionization and preferably protonation may for instance be achieved by providing a suitable pH, e.g. in the binding mixture in order to bring the ionizable group in a charged form, e.g. positively-charged.

According to one embodiment, the ionisable groups of the anion exchange groups are provided on the surface of the particles as monomers, oligomers or polymers. It may be advantageous to provide monomers and oligomers in order to allow eluting bound extracellular RNA in step (ff) using a low salt elution solution, e.g. comprising a total salt concentration of less than 500 mM, such as less then 250 mM, e.g. 200 mM or 10 mM.

According to one embodiment, the particles comprise anion exchange groups that comprise per anion exchange group at least one ionizable group and a linker or spacer moiety. For instance, the anion exchange groups may be attached covalently to the particle, such as magnetic particle. The spacer may then be comprised on the anion exchange groups between the covalent attachment and the ionizable group.

According to a preferred embodiment, the particles comprise anion exchange groups that comprise at least one amino group as ionizable group. Suitable amino groups that can be ionized, in particular by protonation are disclosed below and are known in the field. In particular embodiment, the anion exchange groups comprise one amino group as ionizable group per anion exchange groups. Alternatively, multiple amino groups as ionizable groups are comprised in one anion exchange group.

According to a preferred embodiment, the particles comprise anion exchange groups that comprise at least one primary, secondary or tertiary amino group. In particular, the anion exchange group may comprise a secondary amino group.

According to one embodiment, the anion exchange group of the particles comprises a group selected from the group consisting of primary, secondary and tertiary amines of the formula

(R)₃N, (R)₂NH, RNH₂ and/or X—(CH₂)ₙ—Y
wherein
X is (R)₂N, RNH or NH₂,
Y is (R)₂N, RNH or NH₂,
R is independently of each other a optionally substituted linear, branched or cyclic alkyl, alkenyl, alkynyl or aryl substituent which may comprise one or more heteroatoms, preferably selected from O, N, S and P, and
n is an integer in the range of from 0 to 20, preferably 0 to 18.

According to one embodiment, the anion exchange groups comprise at least one amino group, wherein the amino group is part of a heterocyclic or heteroaromatic ring. In a particular embodiment, the amino group is part of an imidazole ring. Preferably, the anion exchange groups comprise histidine or histamine, or derivatives thereof.

According to one embodiment, the anion exchange groups comprise ionizable groups having a pKa value of the ionized form, preferably the protonated form, of ≤8.0 or ≤7.5.

According to a preferred embodiment, the anion exchange groups comprise ionizable groups having a pKa value of the ionized form, preferably the protonated form, selected from the range of 4.0 to 8.0 or 4.5 to 7.5, preferably 5.0 to 7.5, such as 5.5 to 7.5 or 5.5 to 7.0. Accordingly, the anion exchange groups are preferably positively charged in the binding mixture, having for example a pH of 3 to 5. Moreover, a relatively low pKa value of the anion exchange group advantageously can allow eluting the bound extracellular RNA using high salt, or preferably using low salt elution solutions at a moderate pH, such as 7 to 9.

According to a preferred embodiment, the anion exchange groups comprise at least one ionizable group, wherein said group is ionizable by protonation, wherein the ionizable group is protonated at the acidic pH of the binding mixture of step (aa) and is neutral or uncharged at a basic pH, such as at a basic pH of at least 8, or at least 8.5.

According to a preferred embodiment, the particles comprise anion exchange groups that have a single positive charge per anion exchange group at the pH of the binding mixture of step (aa), optionally at a pH ranging from ≥3 to ≤6 or ≥3.5 ≤5.5.

According to one embodiment, the anion exchange groups comprise a number n ionizable groups per anion exchange group, wherein said number n is selected from the range of 1 to 300, 1 to 250 or 1 to 200, optionally wherein the anion exchange groups are selected from the following group of anion exchange groups:
(i) the anion exchange groups comprise 30 to 300 or 50 to 250 ionizable groups per anion exchange group, optionally wherein these anion exchange groups are provided by poly-histidine;
(ii) the anion exchange groups comprise 2 to <30 ionizable groups per anion exchange group, such as 3 to 25, 4 to 20, 5 to 18, 6 to 15 or 8 to 12 ionizable groups per anion exchange group, optionally wherein these anion exchange groups are provided by oligo-histidine groups; or
(iii) the anion exchange groups comprise 1 to 5 ionizable groups per anion exchange group, such as 1 to 4 or 1 to 3 ionizable groups per anion exchange group, in particular 1, 2 or 3 ionizable groups per anion exchange group.

According to one embodiment, the ionisable groups are provided by amino groups. Accordingly, the ionisable amino groups may be comprised in a polymer, optionally wherein the polymer is (i) a polyalkylimine, optionally selected from polyethylenimine, polypropylenimine, or polybutylenimine, preferably polyethylenimine, or (ii) is a polymer comprising imidazole groups, such as polyhistidine. Such polymers may advantageously allow to bind the extracellular RNA with high efficiency.

According to a particular embodiment, the anion exchange groups of the particles used in step (aa) are selected from (i) polyethyleneimine; (ii) polyhistidine, wherein the number of histidine monomers is at least 30; (iii) oligo-histidine, wherein the number of histidine monomers is in the range of 4 to 18, such as 5 to 16, 6 to 14, 7 to 13 or preferably 8 to 12, and (iv) histamine.

As disclosed therein, the anion exchange groups may comprise at least one amino group that is part of a heterocyclic or heteroaromatic ring. The amino group may be part of an imidazole ring. The anion exchange groups may comprise e.g. histidine and/or histamine. According to one embodiment, the solid phase comprises histamine coupled to a carboxy-modified surface. Alternatively, an imidazole carboxylic acid, such as 4-imidazole acetic acid may be coupled to a surface, such as an amino-modified surface.

According to a particular embodiment, the anion exchange groups comprise histidine or histamine. The number of histidine groups is preferably at least 3 or at least 4. According to one embodiment, the anion exchange groups are selected from (i) oligo-histidine, wherein the number of histidine monomers is in the range of 4 to 18, such as 5 to 16, 6 to 14, 7 to 13 or preferably 8 to 12, and (ii) a histamine group, optionally wherein the anion exchange groups comprise 1 histamine group per anion exchange group.

According to a particular embodiment, the anion exchange groups are selected from (i) polyhistidine and (ii) anion exchange groups comprising Bis-Tris groups. According to one embodiment the number of histidine monomers in the polyhistidine is at least 30.

According to one embodiment, the ionisable amino groups are comprised in a polyalkylimine polymer, optionally selected from polyethylenimine, polypropylenimine, or polybutylenimine, preferably polyethylenimine.

According to one embodiment, the anion exchange groups have a molecular weight per anion exchange group of 1500 Da or less, 1000 Da or less, 500 Da or less or 300 Da or less. According to another embodiment, the anion exchange groups have a molecular weight per anion exchange group of less than 35000 Da or less than 30000 Da. Other molecular weights of anion exchange groups may be used known in the art.

Step (gg)

According to one embodiment, the method further comprises step (gg), analyzing eluted RNA molecules.

The enriched extracellular RNA can be analysed and/or further processed using suitable assay and/or analytical methods. Hence, according to one embodiment, the isolated extracellular nucleic acids are analysed. The analysis can be performed in order to identify, detect, screen for, monitor or exclude a disease, an infection and/or at least one fetal characteristic. The enriched extracellular (total) RNA and/or a specific target extracellular RNA comprised or suspected of being comprised in the enriched isolate can be identified, quantified, modified, contacted with at least one enzyme, amplified, reverse transcribed, cloned, sequenced, contacted with a probe and/or be detected. Respective methods are well-known in the prior art and are commonly applied in the medical, diagnostic and/or prognostic field.

Further Steps

As also disclosed herein the method may also comprise one or more intermediate washing steps.

Automation

According to a preferred embodiment, magnetic anion exchange particles are used for binding the extracellular vesicles in step (aa) and wherein one or more steps of the method are performed using an automated system that moves the magnetic particles by the aid of a magnetic field, optionally wherein steps (aa) to (dd) are performed in an automated manner. It may also be possible to automate particular steps of the method according to the first aspect and perform other steps manually. For instance, optionally, step (aa) may be performed manually and steps (bb) to (dd) or even step (ee) and (ff) may be performed in an automated manner.

### Further Embodiments

Embodiments of the present invention are described again and in further detail in the following. The present invention in particular discloses and provides for the following items:

1. A method for enriching extracellular nucleic acids, such as preferably extracellular RNA from a sample comprising extracellular vesicles, the method comprising the following steps:

(aa) preparing an acidic binding mixture comprising the sample and an anion exchange solid phase which is preferably provided by anion exchange particles and binding extracellular vesicles to the anion exchange solid phase;

(bb) separating the anion exchange solid phase comprising the bound extracellular vesicles from the binding mixture;

(cc) lysing the bound extracellular vesicles in the presence of at least one detergent and binding released nucleic acids, such as preferably RNA to the anion exchange solid phase;

(dd) separating the anion exchange solid phase with the bound nucleic acids from the lysate.

2. The method according to items 1, wherein the method comprises

(ee) optionally washing the bound RNA; and

(ff) eluting the bound RNA from the anion exchange particles.

3. The method according to items 1, wherein step (cc) comprises preparing a lysis mixture by contacting the separated anion exchange particles comprising the bound extracellular vesicles with an acidic lysis reagent which comprises the at least one detergent.

4. The method according to items 3, wherein the detergent is suitable to lyse extracellular vesicles and wherein said detergent is used in the lysis mixture of (cc) in a concentration so that lysis of the bound extracellular vesicles occurs and vesicular RNA is released.

5. The method according to any one of items 1 to 4, wherein in step (cc) the lysis mixture comprising the anion exchange particles comprises the detergent in a concentration of at least 0.1%, such as at least 0.2%, at least 0.5%, at least 0.75% or at least 1%.

6. The method according to any one of items 1 to 4, wherein in step (cc) the lysis mixture comprising the anion exchange particles comprises the detergent in a concentration of at least 1.25%, such as at least 1.5%, at least 1.75% or at least 2%.

7. The method according to any one of items 1 to 6, wherein in step (cc) the lysis mixture comprising the anion exchange particles comprises the detergent in a concentration of 15% or less, 10% or less, 7% or less or 5% or less.

8. The method according to any one of items 1 to 7, wherein step (cc) comprises preparing a lysis mixture by contacting the separated anion exchange particles comprising the bound extracellular vesicles with an acidic lysis reagent which comprises the detergent, wherein the detergent is comprised in the lysis mixture in a concentration in a range of 0.1 to 15%, such as 0.5 to 10%, 0.75% to 7% or 1% to 5%.

9. The method according to one or more of items 1 to 8, wherein the detergent used in step (cc) for lysing the extracellular vesicles is not a cationic detergent.

10. The method according to one or more of items 1 to 9, wherein the at least one detergent used in step (cc) for lysing the extracellular vesicles is selected from a non-ionic surfactant and an anionic detergent.

11. The method according to items 10, wherein the detergent used in step (cc) for lysing the extracellular vesicles is a non-ionic detergent, preferably a polyoxyethylene-based non-ionic detergent.

12. The method according to items 11, wherein the non-ionic detergent is selected from the group consisting of (i) polyoxyethylene fatty alcohol ethers, (ii) polyoxyethylene alkylphenyl ethers, (iii) polyoxyethylene-polyoxypropylene block copolymers, (iv) polyoxyethylene fatty acid esters, (v) ethoxylated propoxylated alcohols, (vi) steroidglycoside-based non-ionic detergents and (vii) sorbitan fatty acid esters,

optionally wherein the non-ionic detergent has at least one of the following characteristics:
(i) it is a polyoxyethylene fatty alcohol ether, optionally comprising a fatty alcohol component having 4 to 28 carbon atoms, and a polyoxyethylene component having 2 to 150 (CH2CH2O) units, optionally selected from a polyoxyethylene lauryl ether, such as polyoxyethylene(4) lauryl ether (e.g. Brij® 30) or polyoxyethylene(23) lauryl ether (e.g. Brij® 35), a polyoxyethylene cetyl ether, such as polyoxyethylene(10) cetyl ether (e.g. Brij® 56) or polyoxyethylene(20) cetyl ether (e.g. Brij® 58), a polyoxyethylene stearyl ether, such as polyoxyethylene(2) stearyl ether (e.g. Brij® 72) or a polyoxyethylene(20) stearyl ether (e.g. Brij® 78), and a polyoxyethylene oleyl ether, such as polyoxyethylene(20) oleyl ether (e.g. Brij® 98);
(ii) it is a polyoxyethylene alkylphenyl ethers, optionally a polyoxyethylene octylphenyl ethers or polyoxyethylene nonylphenyl ethers, optionally branched, optionally selected from a polyoxyethylene p-isooctylphenyl ether (e.g. Triton™ X-100), a polyoxyethylene tert-octylphenyl ether (e.g. Triton™ X-114), a polyoxyethylene (40) isooctylphenyl ether (e.g. Triton™ X-450), a octylphenoxy poly(ethyleneoxy)ethanol (e.g. Igepal® CA-630) or a 4-Nonylphenyl-polyethylene glycol;
(iii) it is a polyoxyethylene-polyoxypropylene block copolymer, such as a poloxamer;
(iv) it is a polyoxyethylene fatty acid ester, such as polyoxyethylene sorbitan monolaurate (Tween® 20), polyoxyethylene sorbitan monooleate (Tween® 80);
(v) it is a ethoxylated propoxylated alcohol, such as seed oil alcohol ethoxylates, in particular seed oil alcohol ethoxylates 4 EO (ECOSURF™ SA-4), seed oil alcohol ethoxylate 7 EO (ECOSURF™ SA-7) or seed oil alcohol ethoxylate 9 EO (ECOSURF™ SA-9);
(vi) it is a steroidglycoside-based non-ionic detergent, such as Digitonin;
(vii) it is a sorbitan fatty acid ester, such as sorbitan monolaurate (e.g. Span® 20), sorbitan monostearate (e.g. Span® 60) or sorbitan monooleate (e.g. Span® 80).

13. The method according to items 10, wherein the detergent used in step (cc) for lysing the extracellular vesicles is an anionic detergent, optionally a sulfate or sulfonate of a fatty alcohol.

14. The method according to items 13, wherein the anionic detergent is selected from the group consisting of
(i) a sulfate or sulfonate of a fatty alcohol, such as sodium dodecyl sulfate, sodium dodecyl sulfonate or dodecylbenzenesulfonic acid;
(ii) a bile-acid based detergent, such as deoxycholate, in particular sodium deoxycholate, or sodium cholate, and
(iii) a sarcosine-based detergent, such as sarkosyl or N-lauroylsarcosine;
optionally wherein the anionic detergent is selected from the group of sodium dodecyl sulfate, sodium dodecyl sulfonate, dodecylbenzenesulfonic acid, N-lauroylsarcosine and sodium cholate, and wherein the anionic detergent optionally is sodium dodecyl sulfate.

15. The method according to any one of items 1 to 14, wherein the detergent used for extracellular vesicle lysis in step (cc) is selected from the group of Triton X-100, sodium dodecyl sulfate, deoxacholate, sarcosyl and/or Ecosurf SA-9.

16. The method according to any one of items 3 to 15, wherein the acidic lysis reagent of step (cc) comprises

(i) the at least one detergent, optionally in a concentration as defined in any one of items 5 to 8 for the lysis mixture; and

(ii) a buffering agent.

17. The method according to items 16, wherein the acidic lysis reagent has an acidic pH that promotes binding of the released vesicular RNA to the anion exchange groups of the particles.

18. The method according to any one of items 13 to 17, wherein the acidic lysis reagent has a pH in the range of 2.5 to 5.5, such as 2.7 to 5.3, 3 to 5 or 3 to 4.7, optionally wherein the pH is in a range of 3 to 4.5 or 3 to 4.3.

19. The method according to items 18, wherein the pH of the acidic lysis reagent is ≤5, optionally ≤4.7, ≤4.5 or ≤4.3.

20. The method according to any one of items 16 to 19, wherein the acidic lysis reagent used in step (cc) has one or more of the following characteristics:
it comprises a carboxylic acid based buffering agent, optionally acetate;
it comprises the buffering agent in a concentration of ≤500 mM, such as ≤450 mM, ≤400 mM, ≤350 mM, preferably ≤300 mM or ≤250 mM.

21. The method according to any one of items 3 to 20, wherein the acidic lysis reagent establishes conditions that allow direct binding of the released vesicular RNA to the anion exchange particles.

22. The method according to any one of items 16 to 21, wherein the total salt concentration in the acidic lysis reagent is 1M or less, preferably 0.75M or less, 0.5M or less or 370 mM or less.

23. The method according to item 22, wherein the total salt concentration in the acidic lysis reagent is 350 mM or less, such as 325 mM or less, 300 mM or less or 275 mM or less.

24. The method according to any one of items 16 to 23, wherein the acidic lysis reagent used in step (cc) has at least one of the following characteristics:
it does not comprise a chaotropic salt; and/or
it does not comprise an organic solvent.

25. The method according to any one of items 1 to 24, wherein step (cc) comprises adding a protease.

26. The method according to any one of items 1 to 25, wherein the protease is a proteinase, preferably proteinase K.

27. The method according to one or more of items 3 to 26, wherein under the conditions established by the acidic lysis reagent agent in step (cc), vesicular RNA that is released from the lysed extracellular vesicles binds to the anion exchange particles present in the lysis mixture.

28. The method according to any one of items 3 to 27, wherein step (cc) comprises contacting the separated anion exchange particles comprising the bound extracellular vesicles with the acidic lysis reagent and wherein no further reagents are added to establish the EV lysis and vesicular RNA binding conditions in step (cc).

29. The method according to any one of items 1 to 28, wherein step (cc) comprises incubating the lysis mixture to allow lysis of extracellular vesicles and direct binding of the released vesicular RNA to the anion exchange particles.

30. The method according to items 29, wherein incubation is performed at room temperature or above.

31. The method according to any one of items 1 to 30, wherein the anion exchange particles that are separated in step (dd) from the lysate comprise bound thereto extracellular RNA which comprises vesicular RNA and optionally non-vesicular RNA.

32. The method according to items 31, wherein the bound RNA comprises non-vesicular RNA that was bound together with the extracellular vesicles in step (aa) to the anion exchange particles.

33. The method according to any one of items 1 to 32, wherein the acidic binding mixture prepared in step (aa) has a pH in the range of 2 to 6, such as 2.5 to 5.5, preferably 3 to 5, more preferably 3 to 4.5.

34. The method according to any one of items 1 to 33, wherein preparing the EV binding conditions in step (aa) comprises adding an acidic reagent, optionally wherein the pH of the acidic reagent is in the range of 2 to 5, such as 2.5 to 5, preferably 3 to 5, more preferably 3 to 4.5.

35. The method according to items 34, wherein the acidic reagent comprises a buffering agent, preferably a carboxylic acid based buffer, optionally wherein the carboxylic acid based buffer has one or more of the following characteristics:
the carboxylic acid based buffering agent comprises a carboxylic acid and a salt of said carboxylic acid, wherein preferably the carboxylic acid (i) comprises 1 to 3 carboxylic acid groups, (ii) is aliphatic, and/or (iii) is saturated;
the carboxylic acid based buffering agent comprises 1 carboxyl group; and/or
the carboxylic acid based buffer is an acetate buffer, optionally provided by a sodium acetate/acetic acid buffer.

36. The method according to items 34 or 35, wherein the EV binding mixture in step (aa) comprises the buffering agent from the acidic reagent in a concentration of 100 mM to 1M, preferably <1M, such as 200 mM to 700 mM, 300 mM to 600 mM or 350 mM to 550 mM, optionally wherein the buffering agent is acetate.

37. The method according to one or more of items 1 to 36, wherein in step (aa) the pH of the binding mixture for binding extracellular vesicles to the anion exchange particles is lower than the pKa of the ionized form of the anion exchange groups of the particles, optionally wherein the pH is at least 1, at least 1.5, at least 2 or at least 2.5 unit(s) lower than the pKa.

38. The method according to one or more of items 1 to 37, wherein in step (aa) the pH of the binding mixture corresponds to the pH of the acidic reagent that is added to adjust the binding conditions or deviates by ≤1, ≤0.75 or preferably ≤0.5 pH units therefrom.

39. The method according to one or more of items 1 to 38, wherein magnetic anion exchange particles are used in step (aa).

40. The method according to one or more of items 1 to 39, wherein the anion exchange particles have one or more of the following characteristics:

(i) they comprise anion exchange groups at the surface of the particles;

(ii) they comprise anion exchange groups of the same or different types;

(iii) the anion exchange groups are attached to the surface of the particles by covalent attachment, optionally using carbodiimide-based reactions, in particular by reacting carboxyl groups of the particles with amino groups comprised in the anion exchange groups.

41. The method according to one or more of items 1 to 40, wherein the anion exchange groups comprise at least one ionizable group as functional group, wherein preferably the ionizable group is ionizable by protonation.

42. The method according to one or more of items 1 to 41, wherein the ionisable groups of the anion exchange groups are provided on the surface of the particles as monomers, oligomers or polymers.

43. The method according to one or more of items 1 to 42, wherein the particles comprise anion exchange groups that comprise per anion exchange group at least one ionizable group and a linker or spacer moiety.

44. The method according to one or more of items 1 to 43, wherein the particles comprise anion exchange groups that comprise at least one amino group as ionizable group.

45. The method according to one or more of items 1 to 44, wherein the particles comprise anion exchange groups that comprise at least one primary, secondary or tertiary amino group.

46. The method according to one or more of items 1 to 45, wherein the anion exchange group of the particles comprises a group selected from the group consisting of primary, secondary and tertiary amines of the formula

(R)₃N, (R)₂NH, RNH₂ and/or X—(CH₂)ₙ—Y

wherein

X is (R)₂N, RNH or NH₂,

Y is (R)₂N, RNH or NH₂,

R is independently of each other a optionally substituted linear, branched or cyclic alkyl, alkenyl, alkynyl or aryl substituent which may comprise one or more heteroatoms, preferably selected from O, N, S and P, and

n is an integer in the range of from 0 to 20, preferably 0 to 18.

47. The method according to one or more of items 1 to 46, wherein the anion exchange groups comprise at least one amino group, wherein the amino group is part of a heterocyclic or heteroaromatic ring.

48. The method according to items 47, wherein the amino group is part of an imidazole ring.

49. The method according to items 48, wherein the anion exchange groups comprise histidine or histamine or derivatives of the foregoing capable of binding extracellular nucleic acids and EVs.

50. The method according to any one of items 1 to 49, wherein the anion exchange groups comprise ionizable groups having a pKa value of the ionized form, preferably the protonated form, of ≤8.0 or ≤7.5.

51. The method according to items 1, wherein the anion exchange groups comprise ionizable groups having a pKa value of the ionized form, preferably the protonated form, selected from the range of 4.0 to 8.0 or 4.5 to 7.5, preferably 5.0 to 7.5, such as 5.5 to 7.5 or 5.5 to 7.0.

52. The method according to one or more of items 1 to 51, wherein the anion exchange groups comprise at least one ionizable group, wherein said group is ionizable by protonation, wherein the ionizable group is protonated at the acidic pH of the binding mixture of step (aa) and is neutral or uncharged at a basic pH, such as at a basic pH of at least 8, or at least 8.5.

53. The method according to one or more of items 1 to 52, wherein the particles comprise anion exchange groups that have a single positive charge per anion exchange group at the pH of the binding mixture of step (aa) and/or step (cc), optionally at a pH ranging from ≥3 to ≤6 or ≥3.5 ≤5.5.

54. The method according to any one of items 1 to 53, wherein the anion exchange groups comprise a number n ionizable groups per anion exchange group, wherein said number n is selected from the range of 1 to 300, 1 to 250 or 1 to 200, optionally wherein the anion exchange groups are selected from the following group of anion exchange groups:
(i) the anion exchange groups comprise 30 to 300 or 50 to 250 ionizable groups per anion exchange group, optionally wherein these anion exchange groups are provided by poly-histidine;
(ii) the anion exchange groups comprise 2 to <30 ionizable groups per anion exchange group, such as 3 to 25, 4 to 20, 5 to 18, 6 to 15 or 8 to 12 ionizable groups per anion exchange group, optionally wherein these anion exchange groups are provided by oligo-histidine groups; or
(iii) the anion exchange groups comprise 1 to 5 ionizable groups per anion exchange group, such as 1 to 4 or 1 to 3 ionizable groups per anion exchange group, in particular 1, 2 or 3 ionizable groups per anion exchange group.

55. The method according to items 54, wherein the ionisable groups are provided by amino groups.

56. The method according to items 55, wherein the ionisable amino groups are comprised in a polymer, optionally wherein the polymer is

(i) a polyalkylimine, optionally selected from polyethylenimine, polypropylenimine, or polybutylenimine, preferably polyethylenimine), or

(ii) is a polymer comprising imidazole groups, such as polyhistidine.

57. The method according to any one of items 1 to 56, wherein the anion exchange groups of the particles used in step (aa) are selected from (i) polyethyleneimine; (ii) polyhistidine, wherein the number of histidine monomers is at least 30; (iii) oligo-histidine, wherein the number of histidine monomers is in the range of 4 to 18, such as 5 to 16, 6 to 14, 7 to 13 or preferably 8 to 12, and (iv) histamine.

58. The method according to any one of items 1 to 57, wherein the anion exchange groups have a molecular weight per anion exchange group of 1500 Da or less, 1000 Da or less, 500 Da or less or 300 Da or less.

59. The method according to any one of items 1 to 57, wherein the anion exchange groups have a molecular weight per anion exchange group of less than 35000 Da or less than 30000 Da.

60. The method according to any one of items 2 to 59, wherein elution step (ff) is performed using one or more elution solutions.

61. The method according to items 60, wherein the elution solution has a basic pH, preferably of at least 8.0, at least 8.3 or at least 8.5 and wherein preferably, the pH of the elution solution is ≤9 or <9.

62. The method according to items 60 or 61, wherein the elution solution comprises a buffering agent, optionally selected from TRIS, HEPES, HPPS or an ammonia buffer, preferably TRIS.

63. The method according to any one of items 60 to 62, wherein elution comprises a heating step.

64. The method according to any one of items 60 to 63, wherein the elution solution having the characteristics according to any one of items 61 to 63 is used in combination with anion exchange particles comprising anion exchange groups that release the bound RNA at the elution conditions provided by the elution solution.

65. The method according to items 64, wherein the anion exchange groups comprise per anion exchange group at least one amino group, optionally 1 to 20 or 1 to 15 amino groups.

66. The method according to items 65, wherein the amino group is part of an imidazole ring.

67. The method according to items 66, wherein the anion exchange groups comprise histidine or histamine.

68. The method according to any one of items 64 to 67, wherein the anion exchange groups of the particles are selected from (i) oligo-histidine, wherein the number of histidine monomers is in the range of 4 to 18, such as 5 to 16, 6 to 14, 7 to 13 or preferably 8 to 12, and (ii) a histamine group, optionally wherein the anion exchange groups comprise 1 histamine group per anion exchange group.

69. The method according to any one of items 60 to 68, wherein the total salt concentration in the elution solution is 500 mM or less, such as 250 mM or less, 200 mM or less, 150 mM or less or 100 mM or less, optionally 50 mM or less.

70. The method according to any one of items 60 to 68, wherein the total salt concentration in the elution solution is at least 500 mM, such as at least 750 mM, at least 1M or at least 1.2M.

71. The method according to any one of items 60 to 68, wherein the elution solution is an extraction buffer, optionally wherein the elution solution has at least one of the following characteristics:

(i) it comprises phenol,

(ii) it comprises a chaotropic salt, optionally selected from guanidinium alts, thiocyanate salts, iodide salts, perchlorate salts, trichloroacetate salts and trifluroacetate salts;

optionally wherein the elution solution has a pH of at least 7.5 or at least 8.

72. The method according to any one of items 1 to 71, wherein step (bb) further comprises washing the separated anion exchange particles.

73. The method according to any one of items 1 to 72, wherein in step (bb) and (dd) the anion exchange particles are separated by centrifugation, sedimentation or magnetic separation, wherein preferably magnetic anion exchange particles are used that are separated by aid of a magnetic field.

74. The method according to any one of items 1 to 73, comprising performing one or more wash steps (ee).

75. The method according to any one of items 1 to 74, wherein the method comprises (gg) analyzing eluted RNA molecules.

76. The method according to any one of items 1 to 75, wherein magnetic anion exchange particles are used for binding the extracellular vesicles in step (aa) and wherein one or more steps of the method are performed using an automated system that moves the magnetic particles by the aid of a magnetic field, optionally wherein steps (aa) to (dd) are performed in an automated manner.

77. The method according to any one of items 1 to 76, wherein the sample comprising extracellular vesicles is or is derived from a body fluid.

78. The method according to items 77, wherein the sample is a sample obtained from a body fluid by removing cells.

79. The method according to items 77 or 78, wherein the sample is a cell-free or cell-depleted body fluid sample.

80. The method according to any one of items 77 to 79, wherein the cell-free or cell-depleted body fluid sample is or is derived from the following samples by removing cells: whole blood, plasma, serum, lymphatic fluid, urine, liquor, cerebrospinal fluid, ascites, milk, bronchial lavage, saliva, amniotic fluid, semen/seminal fluid, body secretions, nasal secretions, vaginal secretions, wound secretions and excretions.

81. The method according to any one of items 77 to 80, wherein the sample is selected from plasma, serum and urine, wherein urine is preferably cell-depleted or cell-free urine.

82. The method according to any one of items 1 to 81, wherein prior to step (a) the method comprises removing cells from a body fluid sample, whereby a cell-depleted body fluid sample is provided as sample comprising extracellular vesicles, wherein said sample is contacted in step (aa) with the anion exchange particles and preferably an acidic reagent such as a binding buffer to prepare the acidic binding mixture.

83. The method according to any one of items 1 to 76, wherein the sample is a cell culture supernatant comprising extracellular vesicles.

84. The method according to any one of items 1 to 83, wherein prior to step (aa) a cell-depleted or cell-free biological sample comprising extracellular vesicles is subjected to a DNA depletion step, in particular by binding DNA, such as extracellular DNA, to a particles comprising anion exchange groups, and separating the bound DNA from the binding mixture, whereby a DNA depleted sample comprising extracellular vesicles is provided that is subjected to step (aa) of the method according to any one of the preceding items for binding extracellular vesicles to the anion exchange particles.

85. The method according any one of items 1 to 84, wherein the method comprises

(a) preparing a binding mixture comprising
a biological sample comprising extracellular vesicles, wherein preferably the biological sample is a cell-depleted or cell-free body fluid sample,
anion exchange particles,
an acidic binding buffer comprising a buffering agent,

and binding extracellular DNA to the particles;

(b) separating the particles with the bound extracellular DNA from the binding mixture, wherein the remaining binding mixture provides a sample comprising extracellular vesicles; and

(c) enriching extracellular vesicles from the remaining binding mixture that provides a sample comprising extracellular vesicles using the method comprising steps (aa) to (dd) according to any one of the preceding items, in particular items 1 to 76.

86. A kit for performing the method according to one or more of items 1 to 85, comprising:

(a) anion exchange particles,

(b) an acidic reagent, preferably an acidic buffer;

(c) an acidic lysis reagent, preferably a buffer which is different from the acidic reagent
(b) and comprises a detergent;

(d) optionally, one or more wash solutions; and

(e) optionally, one or more elution solutions.

87. The kit according to items 86, wherein the anion exchange particles are as defined in any one of items 39 to 59.

88. The kit according to items 86 or 87, wherein the acidic reagent which preferably is a buffer is as defined in any one of items 34 to 38.

89. The kit according to any one of items 86 to 88, wherein the acidic lysis reagent which preferably is a buffer comprises a detergent as defined in any one of items 3 to 15.

90. The kit according to items any one of items 86 to 89, wherein the acidic lysis reagent which preferably is a buffer is as defined in any one of items 2 or 16 to 24.

91. The kit according to any one of items 86 to 90, wherein the kit comprises an elution solution, wherein the elution solution is as defined in any one of items 61 to 71 and/or a protease.

This invention is not limited by the exemplary methods and materials disclosed herein, and any methods and materials similar or equivalent to those described herein can be used in the practice or testing of embodiments of this invention. Numeric ranges are inclusive of the numbers defining the range. The headings provided herein are not limitations of the various aspects or embodiments of this invention which can be read by reference to the specification as a whole.

As used in the subject specification, items and claims, the singular forms “a”, “an” and “the” include plural aspects unless the context clearly dictates otherwise. The terms “include,” “have,” “comprise” and their variants are used synonymously and are to be construed as non-limiting. Further components and steps may be present. Throughout the specification, where compositions are described as comprising components or materials, it is additionally contemplated that the compositions can in embodiments also consist essentially of, or consist of, any combination of the recited components or materials, unless described otherwise. Reference to “the disclosure” and “the invention” and the like includes single or multiple aspects taught herein; and so forth. Aspects taught herein are encompassed by the term “invention”.

It is preferred to select and combine preferred embodiments described herein and the specific subject-matter arising from a respective combination of preferred embodiments also belongs to the present disclosure.

### EXAMPLES

It should be understood that the following examples are for illustrative purpose only and are not to be construed as limiting this invention in any manner. The following examples demonstrate that the method according to the present disclosure allows to effectively enrich cfRNA from a biological sample that comprises extracellular vesicles (EVs). The EVs are bound to solid particles comprising anion exchange groups. After binding and preferably washing the bound EVs, the EV content, including vesicular RNA, is released by performing a detergent-based lysis step. Advantageously, the used lysis conditions allow direct binding of the released cfRNA to the anion exchange particles that were used to capture the EVs. The bound cfRNA may then be washed and eluted from the anion exchange particles. This workflow is highly advantageous as it only requires few steps and is suitable for automation by using magnetic anion exchange particles.

### 1. Example 1

### Magnetic Anion Exchange Particles

Magnetic anion exchange particles (also referred to as “magnetic beads”) used in the examples were prepared as follows. Magnetic beads carrying carboxyl surface groups were coupled to different anion exchange groups using carbodiimide-based coupling. Following anion exchange groups were coupled as ligands:
polyethyleneimine (PEI—“AxpH” beads),
poly-histidine (5,000-25,000 g/mol, n=32-160),
His-10 (oligo-histidine, n=10) and
histamine.

Other anion exchange groups may also be used in the method according to the present disclosure. In particular, anion exchange groups comprising a functional group wherein the protonated form has a pK_{A} value around 5.5 to 9, such 6 to 8 can be advantageously used to facilitate subsequent elution of the bound RNA. The anion exchange groups can be coupled as ligands to the magnetic beads, either using the same coupling chemistry as described above or a different coupling chemistry. Suitable coupling strategies are well-known in the art.

### 2. Example 2

### Binding of EVs, Lysis and Re-Binding of RNA

Example 2 demonstrates the workflow that utilizes a detergent based lysis of EVs bound to the anion exchange particles and instant re-binding of released vesicular RNA.

EV Binding

Human plasma (pooled) was used as a core example of a biological sample comprising EVs. 1 ml of human plasma was contacted with 1 ml of an acidic EV binding buffer. The EV binding buffer used in Example 2 comprised an acetate buffer (here: 465 mM) and had a pH below 5 (here: pH 4) and inverted 5 times. 2 ml of said mixture was then added to AxpH or histamine beads (see Example 1) in order to prepare the binding mixture followed by 10 min end-over-end incubation of the binding mixture. The magnetic anion exchange particles with the bound EVs were magnetically separated for 2 min and the supernatant removed. The separated particles were washed with 1 ml wash buffer (e.g. acetate, pH 5 235 mM salt) to remove sample remainders and separated for 2 min. The wash buffer was removed.

EV Lysis and Binding of the Released Vesicular RNA

The EVs bound to the anion exchange beads were lysed in order to release the vesicular RNA by adding 400 μl of an acidic lysis reagent comprising a detergent and a buffering agent (here: 235 mM acetate buffer, 2% Triton X-100 or 2% Ecosurf SA-9, pH 4). To digest potentially contaminating proteins, a protease (here: proteinase K) was added to some samples to support lysis. The samples were incubated 10 min end-over end. As disclosed herein, these EV lysis conditions effectively lyse EVs, whereby the vesicular RNA is released. In addition, the EV lysis conditions promote binding of the released vesicular RNA to the anion exchange groups of the particles because the EV lysis conditions provide suitable RNA binding conditions. Thus, after EV lysis and instant rebinding of the released vesicular RNA, anion exchange particles are obtained with bound cfRNA. The magnetic particles were then separated for 3 min.

For comparison, the detergent based lysis step was omitted for some samples and the anion exchange particles with the bound EVs were directly processed by addition of QIAzol (see below). It serves the purpose to show EV binding to the anion exchange particles. It furthermore allows to determine whether the vesicular RNA is efficiently released and re-bound to the anion exchange beads during the detergent based EV lysis and cfRNA binding step.

QIAzol Based Elution

Afterwards, the anion exchange beads were separated, eluted in QIAzol and RNA isolation was performed according to miRNeasy protocols:

700 μL QIAzol (QIAGEN), a phenol/guanidine based lysis reagent (adjusted to pH 8) was added to the anion exchange particles for elution/lysis of the bound analytes. cfRNA is released under these conditions from the beads. In addition, EVs are lysed under these conditions, whereby vesicular RNA is released (see control where the detergent based lysis step was omitted and the beads with the bound EVs are directly processed). The samples were briefly vortexed followed by 3 min end-over-end incubation. The anion exchange particles were separated for 2 min and the QIAzol eluate/supernatant comprising the cfRNA was transferred into a 2 mL reaction tube. 90 μL chloroform was added, vortexted (20 sec) and incubated for 2-3 min at room temperature. For separating the aqueous phase which comprises the recovered cfRNA, 15 min centrifugation was performed at 12000 g at 4° C. cfRNA was then purified from the eluate using the miRNeasy Micro protocol (QIAGEN). The aqueous phase was thus transferred into a new 2 mL reaction tube followed by addition of 2× volume EtOH (100%) and mixing. The binding mixture was applied to an RNeasy MinElute Spin Column (centrifugation 8000×g 15 sec, loaded twice) followed by washing and elution of the bound cfRNA.

### REFERENCE

For reference, 1 ml aliquots of the same plasma were processed according to the exoRNeasy workflow (QIAGEN) using an exoEasy column, with or without an additional wash step containing 2% Triton or Ecosurf.

Analysis

Isolated mRNA and miRNA was quantified using qPCR assays for EEF2 (mRNA, QuantiTect assay), let-7a and miR-122 (both using miRCURY LNA assays).

Vesicular RNAs: mRNA EEF2, miRNA let-7a; and

non-vesicular RNA: miR-122.

Results

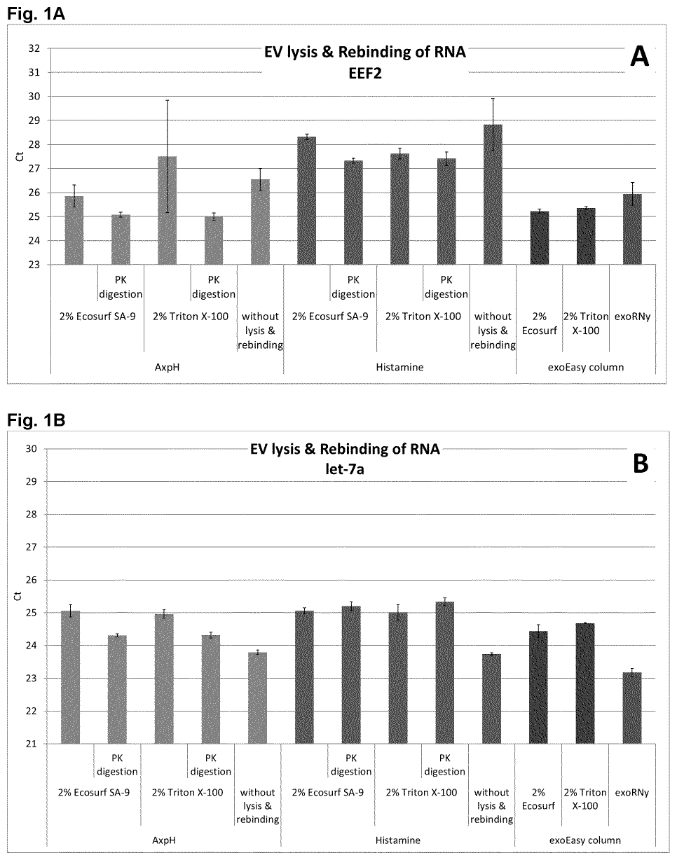

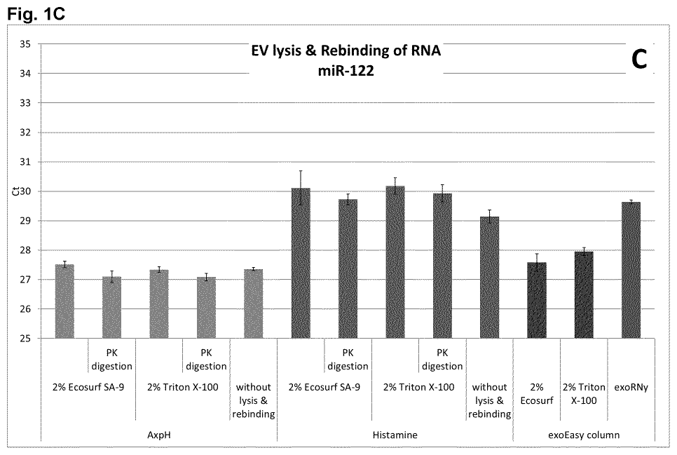

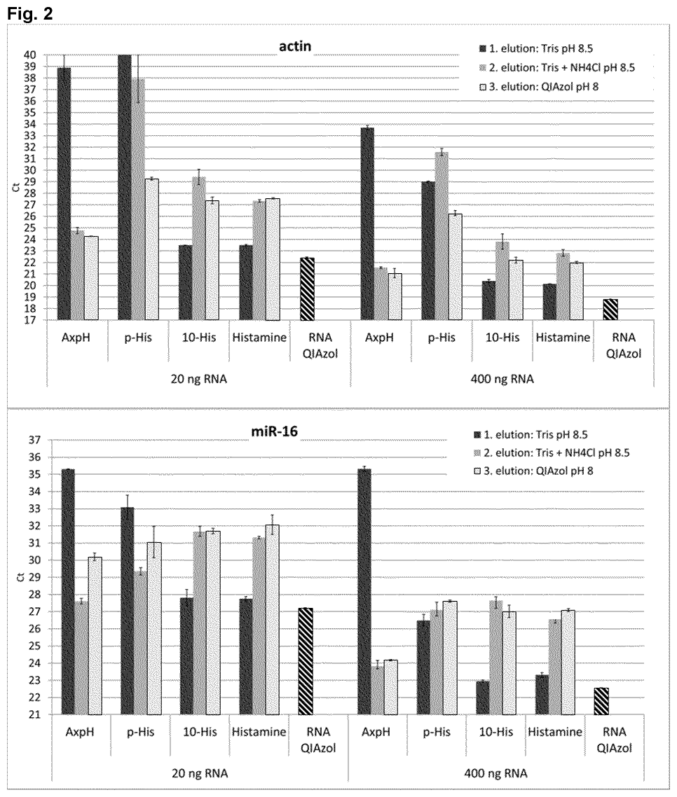

The results are shown in FIG. 1A-C.

The results show that the detergent based lysis step that is used in the methods according to the present disclosure allows to efficiently lyse EVs and bind the released vesicular RNA to the same anion exchange beads that were used for EV binding. mRNA recovery was high.

The results indicate that mRNA was recovered with similar efficiency as exoRNeasy when AxpH beads were used without the additional detergent lysis and rebinding step according to the invention. Binding of EVs to histamine beads was less efficient under the used conditions. Surprisingly, when the detergent based lysis and RNA binding step was included into the workflow, mRNA recovery was not just comparable with the reference exoRNeasy but actually improved for all 3 binding matrices tested, especially when a protease digestion step with proteinase K was included, as well. Thus, vesicular mRNA (EEF2, see FIG. 1A) was recovered with improved efficiency when the detergent based EV lysis and RNA binding step was included. This is an important improvement because vesicular mRNA is an important target analyte.

For let-7a miRNA (FIG. 1B), which occurs primarily inside EVs in plasma from healthy donors, recovery without detergent based lysis was comparable between AxpH and histamine beads, and close to exoRNeasy. When the detergent based lysis was included, recovery actually dropped slightly, indicating that the binding conditions for the short miRNA to the anion exchange beads need further refinement. This can be achieved either by modifying the used EV lysis buffer to establish during lysis conditions that also allow efficient binding of the short miRNA. Alternatively, a binding reagent can be added after EV lysis to improve the binding conditions to promote binding of the released miRNA.

For miR-122 (FIG. 1C), which occurs mainly outside EVs, and is therefore not recovered efficiently using exoRNeasy, recovery using histamine beads was comparable to exoRNeasy and dropped slightly when detergent lysis was included. When using AxpH beads, the recovery was much higher, indicating that not only EVs were captured during the EV binding step, but additionally free Ago2-miRNA complexes that were present in the plasma sample. Therefore, the methods according to the present disclosure also allow to recover non-vesicular RNA, in addition to vesicular RNA. Thus, also total cfRNA can be advantageously isolated.

Overall, the detergent based lysis step allows to efficiently lyse EVs bound to the anion exchange particles and at the same time enables a direct binding of the released vesicular RNA to the same anion exchange particles that were used for binding the EVs. For all three binding matrices tested, the yield of the vesicular mRNA EEF2 was improved with the detergent based lysis step. Lysis of the EVs and recovery of the RNA may be improved by supporting lysis with a protease digestion step (e.g. using proteinase K) as is demonstrated by the yields even further.

### 3. Example 3

### Use of Different Elution Solutions for Different Anion Exchange Beads

Example 2 shows that the method according to the present disclosure provides an efficient workflow for enriching vesicular RNA (and optionally non-vesicular RNA) that only requires few steps to achieve binding of the vesicular RNA/cfRNA to the anion exchange phase. This, and the possibility to use magnetic anion exchange particles, makes the method according to the invention particularly suitable for automation. The provided anion exchange particles with the bound cfRNA may then be washed to remove sample remainders and the cfRNA may then be eluted. To reduce hands-on time and to provide a method that is suitable for automation including a final elution step, an elution step that requires less steps and is more simple than the QIAzol based workflow of Example 2 is desirable. This can be achieved by performing an elution step wherein the anion exchange particles with the bound RNA are contacted with an elution solution at a basic pH.

Example 3 verifies that RNA that is bound to the anion exchange beads can be eluted at moderate pH. To analyze different elution conditions, previously isolated RNA was used as test sample. 20 or 400 ng of RNA from rat liver was incubated in 233 mM acetate buffer, pH 4 with 225 μg magnetic beads carrying either PEI, poly-histidine (p-His), oligo-histidine (10-His) or histamine functional groups for binding the RNA to the anion exchange particles. The particles were then washed once using the same acetate buffer and eluted sequentially, with
(1) 100 μl of 200 mM Tris, pH 8.5,
(2) 100 μl of 200 mM Tris, 1 M NH₄Cl pH 8.5, and
(3) 500 μl QIAzol adjusted to pH 8 (QIAzol pH 8 was prepared by adding 150 μl 2M Tris-base, 2 M guanidine thiocyanate to 700 μl QIAzol),

to determine how much of the RNA once bound to the anion exchange particles can be eluted therefrom at each condition (from mildest to harshest condition, i.e. from (1) to (3)).

To eluates (1) and (2), 500 μl QIAzol was added, and from all 3 primary eluates RNA was re-isolated following the standard miRNeasy Micro protocol. For reference, 20 and 400 ng RNA were added to QIAzol directly and isolated following the same workflow.

The isolated RNA was quantified using qPCR assays for β-actin (mRNA, QuantiTect assay) and miR-16 (miRCURY LNA assay).

Results

The results are shown in FIG. 2. Example 3 shows that bound RNA can be successfully eluted from the anion exchange particles by adding different elution solutions. Overall, different anion exchange particles and elution conditions can be advantageously used for the method according to the present disclosure for extracting RNA. The results demonstrate that RNA can be eluted from the tested anion exchange particles using mild or moderate elution conditions.

The results of the qPCR assays for β-actin and miR-16 are shown in FIG. 2, top. RNA elution from PEI-coated beads was not effective at pH 8.5 using low salt buffer. However, RNA was eluted efficiently in presence of 1 M ammonium chloride (some remaining RNA was eluted by QIAzol only). Therefore, elution with Tris with 1 M ammonium chloride at pH 8.5 provided suitable mild elution conditions.

Surprisingly, with p-His beads RNA elution with Tris at pH 8.5 in low salt buffer was ineffective at low RNA input. Eluting with Tris buffer containing 1 M ammonium chloride, pH 8.5 was about 60-80% effective. Thus, harsher elution conditions are preferred here.

RNA was efficiently eluted from 10-His and histamine beads using Tris pH 8.5. Remaining RNA that was eluted with the harsher elution buffers was over 10-fold lower in concentration (indicating by being detected 3 PCR cycles later), substantiating that the RNA was already efficiently eluted with Tris pH 8.5.

In further experiments, it was demonstrated that elution of RNA in can also be achieved using 10 mM Tris. Hence, in an exemplary workflow suitable for automation, the biological sample comprising EVs (e.g. plasma) is contacted with an acidic binding buffer and magnetic anion exchange particles comprising histamine groups for binding of EVs. After washing, the detergent based EV lysis and RNA binding step is performed, preferably in presence of proteinase K (see Example 2). After separating and washing the histamine beads with the bound cfRNA, the bound RNA is eluted using 10 mM Tris, pH 8.5.

1-34. (canceled)

35. A method for enriching extracellular nucleic acids from a sample comprising extracellular vesicles, the method comprising the following steps:
(aa) preparing an acidic binding mixture comprising the sample and an anion exchange solid phase and binding extracellular vesicles to the anion exchange solid phase;
(bb) separating the anion exchange solid phase comprising the bound extracellular vesicles from the binding mixture;
(cc) lysing the bound extracellular vesicles in the presence of at least one detergent to release vesicular nucleic acids and binding the released vesicular nucleic acids to the anion exchange solid phase; and
(dd) separating the anion exchange solid phase with the bound nucleic acids from the lysate.

36. The method according to claim 35, wherein the extracellular nucleic acids are extracellular RNA.

37. The method according to claim 35, wherein the method further comprises:
(ee) optionally washing the bound nucleic acids; and
(ff) eluting the bound nucleic acids from the anion exchange solid phase.

38. The method according to claim 35, wherein step (cc) comprises preparing a lysis mixture by contacting the separated anion exchange solid phase comprising the bound extracellular vesicles with an acidic lysis reagent which comprises the at least one detergent, wherein the detergent is suitable to lyse extracellular vesicles, and wherein said detergent is used in the lysis mixture of (cc) in a concentration such that lysis of the bound extracellular vesicles occurs and vesicular RNA is released.

39. The method according to claim 35, wherein step (cc) comprises preparing a lysis mixture by contacting the separated anion exchange solid phase comprising the bound extracellular vesicles with an acidic lysis reagent which comprises the detergent, and wherein the detergent is comprised in the lysis mixture in a concentration in a range of 0.1 to 15%.

40. The method according to claim 35, wherein the at least one detergent used in step (cc) for lysing the extracellular vesicles is selected from a non-ionic surfactant and an anionic detergent.

41. The method according to claim 38, wherein the acidic lysis reagent of step (cc) comprises:
(i) the at least one detergent; and
(ii) a buffering agent.

42. The method according to claim 41, wherein the acidic lysis reagent has an acidic pH that promotes binding of the released vesicular RNA to anion exchange groups of the particles, and wherein the acidic lysis reagent has a pH in the range of 2.5 to 5.5.

43. The method according to claim 41, wherein the acidic lysis reagent used in step (cc) has one or more of the following characteristics:
(i) it comprises a carboxylic acid based buffering agent;
(ii) it comprises acetate;
(iii) it comprises the buffering agent in a concentration of ≤500 mM;
(iv) it establishes conditions that allow direct binding of the released vesicular RNA to the anion exchange solid phase;
(v) the total salt concentration in the acidic lysis reagent is 1M or less;
(vi) it does not comprise a chaotropic salt; and
(vii) it does not comprise an organic solvent.

44. The method according to claim 35, wherein step (cc) comprises adding a protease.

45. The method according to claim 44, wherein the protease has one or more of the following characteristics:
the protease is a proteinase; and
the protease is proteinase K.

46. The method according to claim 38, wherein the anion solid phase is provided by anion exchange particles, wherein under the conditions established by the acidic lysis reagent agent in step (cc), vesicular RNA that is released from the lysed extracellular vesicles binds to the anion exchange particles present in the lysis mixture, wherein step (cc) comprises contacting the separated anion exchange particles comprising the bound extracellular vesicles with the acidic lysis reagent, wherein no further reagents are added to establish the EV lysis and vesicular RNA binding conditions in step (cc), and wherein the anion exchange particles that are separated in step (dd) from the lysate comprise bound thereto extracellular RNA which comprises vesicular RNA and optionally non-vesicular RNA.

47. The method according to claim 35, wherein the acidic binding mixture prepared in step (aa) has a pH in the range of 2 to 6, and wherein preparing the EV binding conditions in step (aa) comprises adding an acidic reagent.

48. The method according to claim 47, wherein the acidic reagent has one or more of the following characteristics:
the pH of the acidic reagent is in the range of 2 to 5;
it comprises a buffering agent;
it comprises a carboxylic acid based buffer;
it comprises a carboxylic acid based buffer that comprises a carboxylic acid and a salt of said carboxylic acid;
it comprises a carboxylic acid that
(i) comprises 1 to 3 carboxylic acid groups,
(ii) is aliphatic, and/or
(iii) is saturated;
it comprises a carboxylic acid based buffering agent that comprises 1 carboxyl group;
it comprises an acetate buffer; and
it comprises a sodium acetate/acetic acid buffer.

49. The method according to claim 35, wherein the anion solid phase is provided by anion exchange particles, and wherein in step (aa) the pH of the binding mixture for binding extracellular vesicles to the anion exchange particles is lower than the pKa of the ionized form of anion exchange groups of the particles.

50. The method according to claim 35, wherein the pH of the binding mixture in step (aa) is at least 1 unit lower than the pKa of the ionized form of anion exchange groups of the particles.

51. The method according to claim 35, wherein magnetic anion exchange particles are used in step (aa) as anion exchange solid phase.

52. The method according to claim 35, wherein the anion solid phase is provided by anion exchange particles, and wherein the anion exchange particles have one or more of the following characteristics:
(i) they comprise anion exchange groups at the surface of the particles;
(ii) they comprise anion exchange groups of the same or different types;
(iii) anion exchange groups are attached to the surface of the particles by covalent attachment;
(iv) the anion exchange groups are attached to the surface of the particles by using carbodiimide-based reactions; and
(v) the anion exchange groups are attached to the surface of the particles by reacting carboxyl groups of the particles with amino groups comprised in the anion exchange groups.

53. The method according to claim 35, wherein anion exchange groups of the solid phase have one or more of the following characteristics:
(i) they comprise at least one ionizable group as functional group;
(ii) they comprise at least one ionizable group as functional group that is ionizable by protonation;
(iii) they comprise at least one ionizable group as functional group that is provided on the surface of the solid phase as monomers, oligomers or polymers;
(iv) they comprise at least one primary, secondary or tertiary amino group; and
(v) they comprises a group selected from the group consisting of primary, secondary and tertiary amines of the formula
(R)₃N, (R)₂NH, RNH₂ and/or X—(CH₂)ₙ—Y
wherein
X is (R)₂N, RNH or NH₂,
Y is (R)₂N, RNH or NH₂,
R is independently of each other a optionally substituted linear, branched or cyclic alkyl, alkenyl, alkynyl or aryl substituent which may comprise one or more heteroatoms selected from O, N, S and P, and
n is an integer in the range of from 0 to 20.

54. The method according to claim 35, wherein the anion solid phase is provided by anion exchange particles, wherein the anion exchange particles comprise anion exchange groups at the surface of the particles, wherein the anion exchange groups comprise at least one amino group, and wherein the amino group is part of a heterocyclic or heteroaromatic ring.

55. The method according to claim 54, wherein the amino group is part of an imidazole ring.

56. The method according to claim 55, wherein the anion exchange groups comprise at least one of the following characteristics:
they comprise histidine;
they comprise histamine; and
they comprise derivatives of the foregoing capable of binding extracellular nucleic acids and EVs.

57. The method according to claim 35, wherein the anion solid phase is provided by anion exchange particles, wherein the anion exchange particles comprise anion exchange groups at the surface of the particles, wherein the anion exchange groups have one or more of the following characteristics:
(i) they comprise ionizable groups having a pKa value of the ionized form of ≤8.0 or ≤7.5;
(ii) they comprise ionizable groups having a pKa value of the ionized form selected from the range of 4.0 to 8.0;
(iii) they comprise at least one ionizable group, wherein said group is ionizable by protonation, wherein the ionizable group is protonated at the acidic pH of the binding mixture of step (aa) and is neutral or uncharged at a basic pH;
(iv) they comprise at least one ionizable group, wherein said group is ionizable by protonation, wherein the ionizable group is protonated at the acidic pH of the binding mixture of step (aa) and is neutral or uncharged at a pH of at least 8; and
(v) they have a single positive charge per anion exchange group at the pH of the binding mixture of step (aa) and/or step (cc).

58. The method according to claim 35, wherein the anion solid phase is provided by anion exchange particles, wherein the anion exchange particles comprise anion exchange groups at the surface of the particles, wherein the anion exchange groups comprise a number n ionizable groups per anion exchange group, wherein said number n is selected from the range of 1 to 300, and wherein the anion exchange groups are selected from the following group of anion exchange groups:
(i) the anion exchange groups comprise 30 to 300 ionizable groups per anion exchange group;
(ii) the anion exchange groups are provided by poly histidine and comprise 30 to 300 ionizable groups per anion exchange group;
(iii) the anion exchange groups comprise 2 to <30 ionizable groups per anion exchange group;
(iv) the anion exchange groups are provided by oligo-histidine groups and comprise 2 to <30 ionizable groups per anion exchange group; and
(v) the anion exchange groups comprise 1 to 5 ionizable groups per anion exchange group.

59. The method according to claim 58, wherein the ionisable groups are provided by amino groups with at least one of the following characteristics:
(i) the ionizable amino groups are comprised in a polymer;
(i) the ionizable amino groups are comprised in a polyalkylimine;
(iii) the ionizable amino groups are selected from polyethyleneimine, polypropyleneimine or polybutyleneimine;
(iv) the ionizable amino groups are comprised in a polymer comprising imidazole groups;
(v) the ionizable amino groups are comprised in a polymer comprising polyhistidine; and
(vi) the ionizable amino groups are comprised in a polymer comprising oligo histidine.

60. The method according to claim 35, wherein anion exchange particles are used as solid phase, and wherein anion exchange groups of the particles used in step (aa) are selected from:
(i) polyethyleneimine;
(ii) polyhistidine, wherein the number of histidine monomers is at least 30;
(iii) oligo-histidine, wherein the number of histidine monomers is in the range of 4 to 18; and
(iv) histamine.

61. The method according to claim 35, wherein magnetic anion exchange particles are used for binding the extracellular vesicles in step (aa), and wherein one or more steps of the method are performed using an automated system that moves the magnetic particles by the aid of a magnetic field.

62. The method according to claim 35, wherein the sample comprising extracellular vesicles has one or more of the following characteristics:
(i) it is a body fluid or is derived from a body fluid;
(ii) it is a cell-free or cell-depleted body fluid sample;
(iii) it is a sample obtained from a body fluid by removing cells;
(iv) it is a cell-free or cell-depleted body fluid sample that is or is derived from the following samples by removing cells: whole blood, plasma, serum, lymphatic fluid, urine, liquor, cerebrospinal fluid, synovial fluid, interstitial fluid, ascites, milk, bronchial lavage, saliva, amniotic fluid, semen/seminal fluid, body secretions, nasal secretions, vaginal secretions, wound secretions and excretions;
(v) it is selected from plasma, serum and urine;
(v) it is cell-depleted or cell-free urine; and
(vi) it is a cell culture supernatant comprising extracellular vesicles.

63. The method according to claim 35, wherein prior to step (a) the method comprises removing cells from a body fluid sample, whereby a cell-depleted body fluid sample is provided as a sample comprising extracellular vesicles, wherein said sample comprising extracellular vesicles is contacted in step (aa) with the anion exchange solid phase, which is in the form of particles and an acidic reagent to prepare the acidic binding mixture.

64. The method according to claim 35, wherein prior to step (aa) a cell-depleted or cell-free biological sample comprising extracellular vesicles is subjected to a DNA depletion step by binding DNA to particles comprising anion exchange groups, and separating the bound DNA from the binding mixture, whereby a DNA depleted sample comprising extracellular vesicles is provided that is subjected to step (aa) of the method for binding extracellular vesicles to an anion exchange solid phase which is provided by anion exchange particles.

65. The method according to claim 35, wherein the method comprises
(a) preparing a binding mixture comprising
a biological sample comprising extracellular vesicles, wherein the biological sample is a cell-depleted or cell-free body fluid sample,
anion exchange particles,
an acidic binding buffer comprising a buffering agent,
and binding extracellular DNA to the particles;
(b) separating the particles with the bound extracellular DNA from the binding mixture, wherein the remaining binding mixture provides a sample comprising extracellular vesicles; and
(c) enriching extracellular vesicles from the remaining binding mixture that provides a sample comprising extracellular vesicles using the method comprising steps (aa) to (dd).

66. A kit for performing the method according to claim 35, comprising:
(a) anion exchange particles,
(b) an acidic reagent;
(c) an acidic lysis reagent, which is different from the acidic reagent (b) and comprises a detergent;
(d) optionally, one or more wash solutions; and
(e) optionally, one or more elution solutions.

67. The kit according to claim 66, wherein the anion exchange particles are magnetic anion exchange particles.

68. The kit according to claim 66, wherein the acidic reagent (b) is has on or more of the following characteristics:
it has a pH in the range of 2 to 6;
it comprises a buffering agent;
it comprises a carboxylic acid based buffer;
it comprises a carboxylic acid based buffer that comprises a carboxylic acid and a salt of said carboxylic acid;
it comprises a carboxylic acid that
(i) comprises 1 to 3 carboxylic acid groups,
(ii) is aliphatic, and/or
(iii) is saturated;
it comprises a carboxylic acid based buffering agent that comprises 1 carboxyl group;
it comprises an acetate buffer; and
it comprises a sodium acetate/acetic acid buffer.

69. The kit according to claim 66, wherein the acidic lysis reagent (c) comprises a detergent selected from a non-ionic detergent and an anionic detergent.

70. The kit according to claim 66, wherein the acidic lysis reagent (c) is has at least one of the following characteristics:
it comprises at least one detergent, wherein the detergent is suitable to lyse extracellular vesicles, and wherein said detergent is used in the lysis mixture of (cc) in a concentration such that lysis of the bound extracellular vesicles occurs and vesicular RNA is released;
it comprises a detergent, and wherein the detergent is comprised in the lysis mixture in a concentration in a range of 0.1 to 15%;
it comprises at least one detergent which is selected from a non-ionic surfactant and an anionic detergent;
it comprises at least one detergent and a buffering agent; and
it comprises a buffering agent.

71. The kit according to claim 66, wherein the kit comprises an elution solution and/or a protease.
